(19) United States
(12) Patent Application Publication
(10) Pub. No.: US 2026/0157822 A1
(43) Pub. Date: Jun. 11, 2026

(54) METHODS FOR DENTAL APPLIANCE RETENTION MODIFIED STAGING

(71) Applicant: ALIGN TECHNOLOGY, INC., San Jose, CA (US)

(72) Inventors: Iman Shojaei, San Jose, CA (US); Kangning Su, Pullman, WA (US); Tzishing Jesse Lim, Mountain View, CA (US); Yuxiang Wang, Newark, CA (US); Jun Sato, San Jose, CA (US); Eric Yau, Santa Clara, CA (US); Sudharshan Anandan, Sunnyvale, CA (US); Yueping Wang, San Jose, CA (US); Wilson Ting, San Ramon, CA (US); Crystal Tjhia, Sunnyvale, CA (US); John Y. Morton, San Jose, CA (US); Aja Pariante Hartman, Sunnyvale, CA (US); Lisa Danielle Brock, Cupertino, CA (US)

(21) Appl. No.: 19/252,072

(22) Filed: Jun. 27, 2025

Related U.S. Application Data
(60) Provisional application No. 63/666,071, filed on Jun. 28, 2024.
(60) Provisional application No. 63/666,051, filed on Jun. 28, 2024.

(51) Int. Cl. A61C 7/00; A61C 7/08; A61C 13/34; G16H 20/40

(52) U.S. Cl. CPC A61C 7/002; A61C 7/08; A61C 13/34; G16H 20/40

(57) ABSTRACT

A method for designing dental appliances may include receiving a 3D model of a patient's oral cavity and determining a target arrangement of teeth of the patient from the 3D model. A treatment plan for moving teeth of a patient from an initial position towards and final position over a plurality of stages may be generated. For a stage of the plurality of stages, the method may include identifying which of the teeth of the patient are stationary for the stage of the treatment plan, modifying movement of the stationary teeth for the stage of the treatment plan, and revising the treatment plan using a modified movement of the stationary teeth for the stage of the plurality of stages to generate a revised treatment plan.

### RELATED APPLICATIONS

This application claims the benefit under 35 U.S.C. § 119(e) of U.S. Provisional Patent Application No. 63/666,051, filed Jun. 28, 2024, and titled “SYSTEMS AND METHODS FOR DENTAL APPLIANCE RETENTION WITH TOOTH CAVITY DEVIATIONS,” and U.S. Provisional Patent Application No. 63/666,071, filed Jun. 28, 2024, and titled “SYSTEMS AND METHODS FOR DENTAL APPLIANCE RETENTION,” both of which are incorporated herein, in their entirety, by this reference.

### BACKGROUND

Retention of dental appliances on a patient's teeth play a role in applying forces, such as for orthodontic treatment. However the smooth and uniform surfaces reduce the retention between teeth and a dental appliance such as an orthodontic aligner. When applying a movement force to one tooth, a reaction force may be applied to other teeth of the patient's dentition such as teeth adjacent to the moving tooth. When an aligner has low retention on a moving tooth for teeth to which a reaction force is applied, the aligner may not provide the desired movement force to move the teeth during orthodontic or other treatment.

Current methods of aligner retention may be less than ideal for a number of reasons. For example, adding more attachments during orthodontic treatment can increase cost and time for application of the attachments. Attachments may also fall off during treatment resulting in less predictable tooth movements or reapplication of replacement attachments. Attachments may also be visible to others, creating a less than ideal aesthetic for the patient's teeth during treatment.

### SUMMARY

Accordingly, as will be described in greater detail below, the present disclosure describes various systems and methods for treatment planning and dental treatment for increasing the retention of the dental appliance as compared to existing systems and methods. The increase in retention may be achieved without the use of additional attachments. For example, tooth cavity deviations may increase the frictional forces between the aligner and the tooth allowing an aligner to more strongly grip the surface of a tooth that may not have an attachment or without the use of an attachment on the tooth. The systems and methods disclosed herein may be used to generate treatment plans that provide for retention of appliances on a patient's arch during dental treatments, such as orthodontic treatments, and for dental appliances that provide retention on a patient's arch during dental treatments, such as orthodontic treatment.

In addition, the systems and methods described herein may improve the functioning of a computing device and related systems by reducing computing resources and overhead for acquiring scan data and generating three-dimensional models of the patient's dentition for use in generating retention models, treatment plans, in dental appliances, thereby improving processing efficiency of the computing device over conventional approaches. These systems and methods may also improve the field of dental treatment, including prosthodontics and orthodontics, by analyzing data and carrying out methods that lead to more efficient use of dental resources and improved dental treatment using dental appliances.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1 depicts a flow diagram for generating orthodontic aligners with improved retention, in accordance with some embodiments;

FIG. 2 depicts a flow diagram for modeling the retention on a retentive arch, in accordance with some embodiments;

FIG. 3 depicts a flow diagram for modeling the retention on a planned arch, in accordance with some embodiments;

FIG. 4 depicts various views of a retentive arch and applications of forces thereon, in accordance with some embodiments;

FIG. 5 depicts views of a planned arch, in accordance with some embodiments;

FIG. 6 depicts a table showing an estimated retention on a planned arch and a retentive arch, in accordance with some embodiments;

FIGS. 7A, 7B, and 7C depict various modified aligners with features that improve the retention of the aligner on a patient's dentition, in accordance with some embodiments;

FIG. 8 depicts aligners and their relative retention, in accordance with some embodiments;

FIG. 9A depicts aligner-tooth friction, in accordance with some embodiments;

FIG. 9B depicts various options for aligner retention, in accordance with some embodiments;

FIGS. 9C and 9D depict aligner-tooth friction after aligner offset has been applied, in accordance with some embodiments;

FIG. 10 depicts models of aligners with a modified tooth receiving cavity for aligner retention, in accordance with some embodiments;

FIG. 11 depicts a flow diagram for generating orthodontic aligners with improved retention, in accordance with some embodiments;

FIG. 12 depicts a flow diagram for determining which teeth are stationary teeth, in accordance with some embodiments;

FIG. 13 depicts aspects of how to determining which teeth are stationary teeth, in accordance with some embodiments;

FIG. 14A depicts a method for increasing retention with tooth cavity perturbations, in accordance with some embodiments;

FIG. 14B depicts aligner-tooth friction, in accordance with some embodiments;

FIG. 15A depicts a first change in tooth movement, in accordance with some embodiments;

FIG. 15B depicts the first change on the aligner, in accordance with some embodiments;

FIG. 15C depicts a change in friction between the aligner and the tooth based on the first change on the aligner, in accordance with some embodiments;

FIG. 16A depicts a second change in tooth movement, in accordance with some embodiments;

FIG. 16B depicts the second change on the aligner, in accordance with some embodiments;

FIG. 16C depicts a change in friction between the aligner and the tooth based on the second change on the aligner, in accordance with some embodiments;

FIG. 16D depicts three tables shown the planned tooth movements, the planned deviations, and the resulting tooth movements, for a subset of teeth for eight stages of treatment, in accordance with some embodiments;

FIGS. 17A, 17B, 17C, 17D, and 17E depict topographical appliance features, in accordance with some embodiments;

FIGS. 18A, 18B, 18C, and 18D depict topographical appliance feature placement on dental appliances, in accordance with some embodiments;

FIG. 19A depicts an example dental appliance with topographical appliance features, in accordance with some embodiments;

FIG. 19B depicts an example dental appliance with topographical appliance features, in accordance with some embodiments;

FIG. 20 depicts a method of appliance design using topographical appliance features, in accordance with some embodiments;

FIG. 21A depicts a method of appliance design using topographical appliance features, in accordance with some embodiments;

FIG. 21B depicts a method of appliance design using topographical appliance features, in accordance with some embodiments;

FIG. 22 depicts a method of appliance design using topographical appliance features, in accordance with some embodiments;

FIG. 23 depicts simulated results of retention improvement using topographical appliance features discussed herein, in accordance with some embodiments;

FIG. 24 depicts a system for designing appliances in executing methods and processes described herein, in accordance with some embodiments;

FIG. 25 depicts an exemplary tooth repositioning appliance, in accordance with some embodiments;

FIG. 26 depicts a tooth repositioning system, in accordance with some embodiments;

FIG. 27 depicts a method of orthodontic treatment using a plurality of appliances, in accordance with some embodiments;

FIG. 28 depicts a method for planning an orthodontic treatment, in accordance with some embodiments; and

FIG. 29 depicts a block diagram of a data processing system for use in executing methods and processes described herein, in accordance with some embodiments.

### DETAILED DESCRIPTION

The following detailed description and figures provide a better understanding of the features and advantages of the inventions described in the present disclosure in accordance with the embodiments disclosed herein. Although the detailed description and figures include many specific embodiments, these are provided by way of example only and should not be construed as limiting the scope of the inventions disclosed herein.

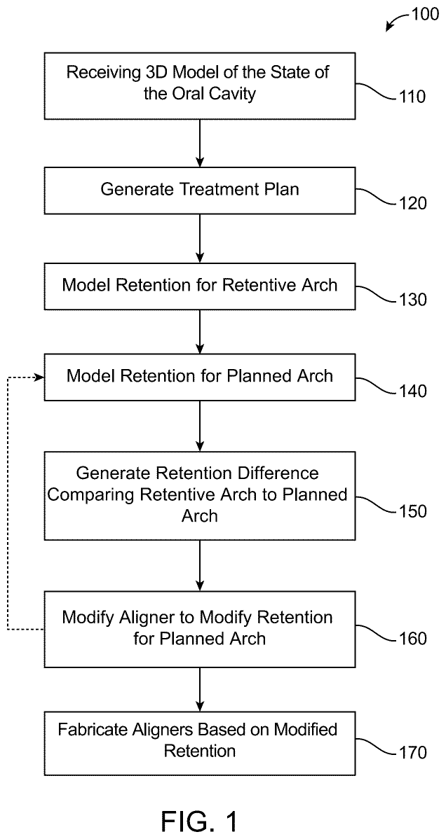

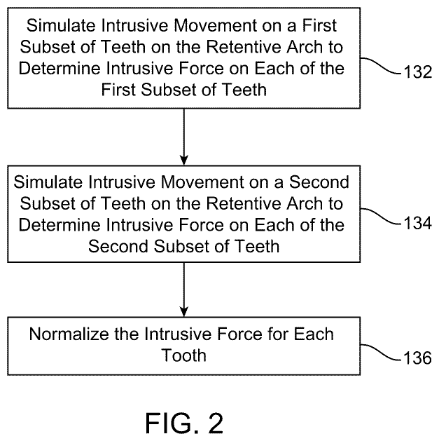

As shown in FIG. 1, an embodiment of a method 100 for designing a retentive dental appliance may include receiving a 3D model of the state of the oral cavity of block 110, generating a treatment plan for the patient at block 120, modeling the retention of the dental appliance on a retentive arch at block 130, modeling retention of the dental appliance on a planned or actual arch at block 140, generating a retention difference comparing the retentive arch to the planned arch at block 150, to modify the retention of the planned or actual arch at block 160.

Increased retention of an orthodontic aligner refers to how securely the aligner fits over the teeth, and this has a direct impact on the effectiveness of the aligner in imparting movement forces to the teeth. When an aligner fits snugly and is well retained on the teeth, it is better able to transfer the designed forces to the teeth and may be able to impart greater tooth movement forces on the teeth. High retention also aids in the aligner's contact with the intended surfaces of the teeth, allowing for precise control over the tooth movement. This is particularly useful for complex movements like rotations and extrusions.

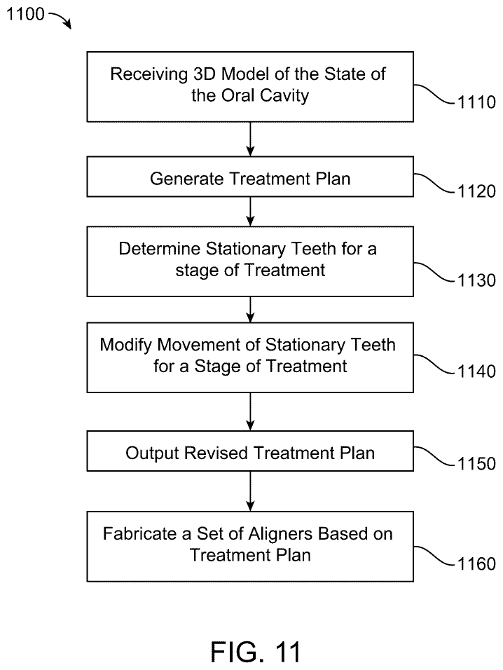

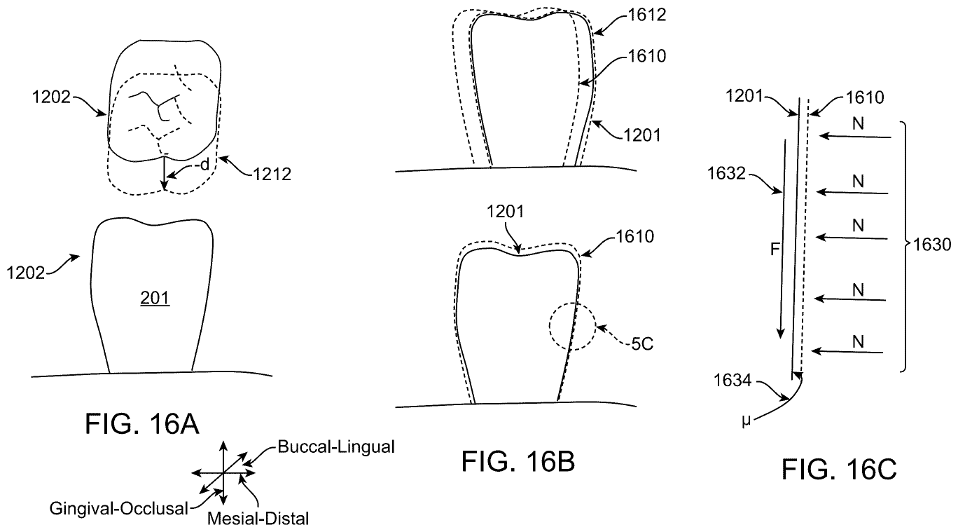

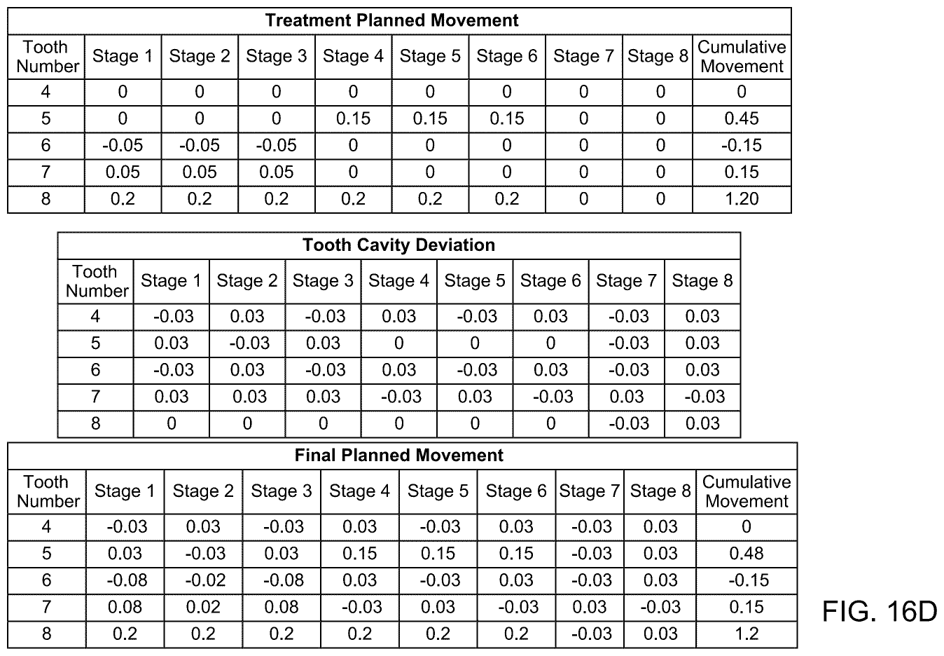

The process shown in FIG. 1 may be performed by any suitable computer-executable code and/or computing system, including the system(s) illustrated in FIGS. 11 and 16. In one example, each of the steps of the process 100 shown in FIG. 1 may represent an algorithm whose structure includes and/or is represented by multiple sub-steps, examples of which will be provided in greater detail below.

At block 110 a 3D model of the state of the patient's oral cavity may be received. The 3D model may be a digital representation and may include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.). The 3D model of the state of the patient's oral cavity may also include subsurface data of the patient's intraoral cavity such as information related to the size, shape, location, and orientation of the roots of the patient's teeth and features of the patient's dental tissues such as the interior of the patient's teeth, the patient's jaws, and the patient's gingiva. Surface typology and subsurface information may be provided by x-rays, CBCT scans, and two-dimensional photos of the patient's dentition and oral cavity.

In some embodiments, at block 110, the patient's oral cavity may be imaged using an intraoral scanner, an x-ray scanner, a CBCT scanner, or other imaging systems such as infrared and visible light images which may be 2D images.

In some embodiments, the patient's dentition may be segmented at block 110. Tooth segmentation of dental scans analyzes and processes images obtained through various dental scanning techniques (such as X-rays, CBCT scans, CT scans, or 3D imaging) to segment tooth to accurately distinguish and separate the different teeth and/or components of the teeth from the surrounding tissue and from each other in these digital images. This separation may be used for diagnosis, treatment planning, orthodontic study, and the creation of dental prosthetics.

At block 120 a treatment plan for moving a patient's teeth from an initial arrangement towards a final arrangement is determined. The patient's initial arrangement may be based on the state of the patient's oral cavity determined at block 110. In some embodiments, at block 120 a final or target arrangement may be determined. The final or target arrangement may be the goal for an arrangement of the patient's teeth at the end of treatment.

A treatment plan may include multiple stages for moving the patient's teeth from an initial arrangement to towards the final arrangement. The treatment stages can be incremental repositioning stages of an orthodontic treatment procedure designed to move one or more of the patient's teeth from an initial tooth arrangement towards a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

For each stage of treatment a digital model of the patient's dentition may be generated and used as a basis for forming an appliance such as an orthodontic aligner for that stage of treatment. In some embodiments treatment planning may also include determining the location shape and orientation of other appliance features such as attachments on the patient's teeth and other aligner features described herein.

At block 130 a model of a retentive arch based on the patient's dentition may be generated. The model of the retentive arch may be a highly retentive arch in which additional attachments or applied to the patient's teeth. For example, the treatment plan may indicate attachments are applied to a subset of the patient's teeth such as four or five of the patient's teeth, however in the highly retentive arch, attachments may be applied to additional teeth. In some embodiments, attachments may be applied to each of the patient's teeth. In some embodiments, multiple attachments may be applied to each of the patient's teeth. In some embodiments provided in the treatment plan may be removed and replaced with attachments having different locations, shapes, or other features. In some embodiments, attachments may be applied to both the buccal and lingual surfaces of each of the patient's teeth.

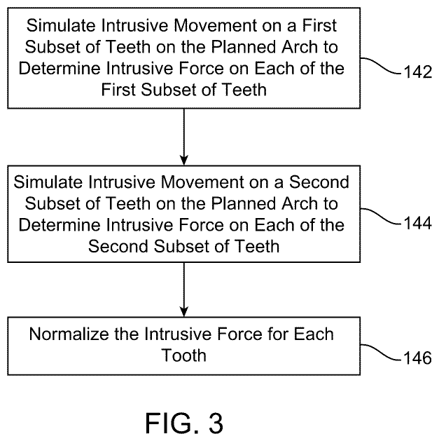

The retention of the aligner on a patient's retentive arch may be modeled as shown and described in FIG. 3. At block 132, an intrusive movement is simulated on a first subset of teeth on the retentive arch to determine the intrusive achieved force on each of the first subset of teeth. The first set of teeth may be the odd-numbered teeth of the patient's dentition. In some embodiments, the first set of teeth may be every other tooth in patient's dentition. Intrusive movement may be a movement of between 0.05 and 2 mm.

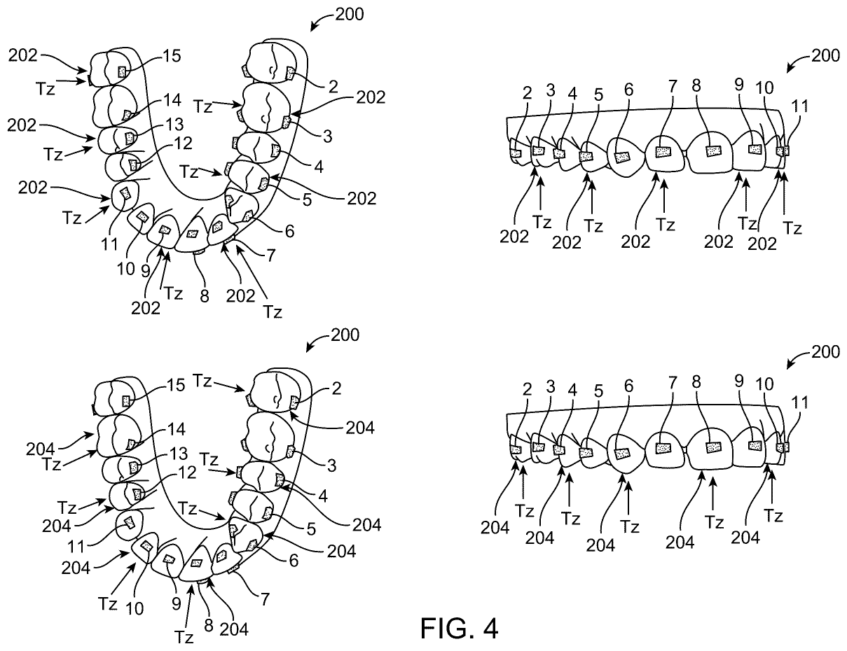

FIG. 4 depicts a retentive arch 200. The first set of teeth may be teeth 202. Simulating the intrusive movement on the patient's teeth may include determining the intrusive force (Fz) imparted on each tooth by an orthodontic appliance shaped to impart the intrusive movement on the patient's teeth. The simulation may be carried out using finite element analysis or other types of digital or numerical simulation of an appliance on the retentive arch 200.

At block 134, an intrusive movement is simulated on a second subset of teeth on the retentive arch to determine the intrusive achieved force on each of the second subset of teeth. The second set of teeth may be the even teeth of the patient's dentition. In some embodiments, the second set of teeth or every other tooth in patient's dentition between each of the teeth in the first set of teeth. Intrusive movement may be a movement of between 0.05 and 2 mm.

FIG. 4 depicts a retentive arch 200. The second set of teeth may be teeth 204. Simulating the intrusive movement on the patient's teeth may include determining the intrusive force (Fz) imparted on each tooth by an orthodontic appliance shaped to impart the intrusive movement on the patient's teeth. The simulation may be carried out using finite element analysis or other types of digital or numerical simulation of an appliance on the retentive arch 200.

At block 136 the intrusive force for each tooth may be normalized. In some embodiments, normalization of the intrusive force may be determined based on the overall intrusive forces applied to the set of teeth. For example when modeling the intrusion on the first set of teeth, the intrusive force on each of the first set of teeth may be determined and then added together to determine the overall intrusive force on the first set of teeth. The normalized force on each of the first set of teeth may then be the force on a particular tooth of the first set of teeth divided by the total force on the first set of teeth.

In some embodiments, the normalized force on each of the second set of teeth may be the force on a particular tooth of the second set of teeth divided by the total intrusive force on the second set of teeth.

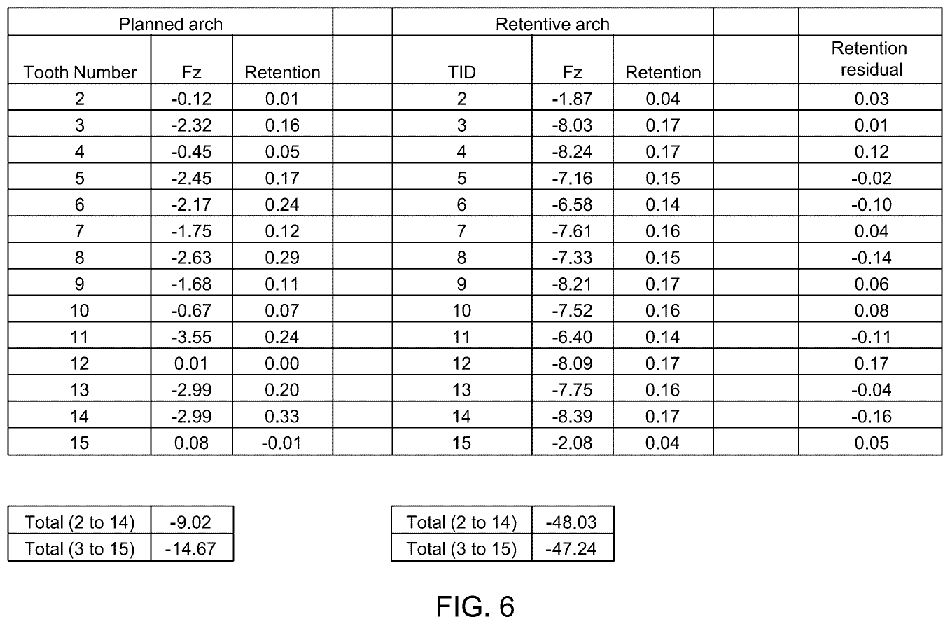

FIG. 6 depicts a table that shows the tooth identification (TID), the intrusive force (Fz) applied to each tooth, and the normalized retention for each tooth of the retentive arch along with total intrusion force on the even-numbered teeth (the first set of teeth), and the total intrusion force on the odd-numbered teeth (the second set of teeth).

In some embodiments, the normalized value for each individual tooth and the total force and the total force on the second set of teeth may be stored for later comparison. The retentive arch, which may be referred to as a highly retentive arch may be used as a control for the two forces and retention provided by an aligner on the actual arch.

At block 140 a model of an actual arch based on the patient's dentition and or the stage of the treatment plan may be generated. The model of the actual arch may include the dental appliances, such as dental attachments as determined by the treatment plan. The model of the actual arch may also include the planned movements of the patient's teeth for a stage of treatment.

The retention of the aligner on a patient's actual arch may be modeled as shown and described in FIG. 3. At block 142, an intrusive movement is simulated on a first subset of teeth 302 on the actual arch to determine the intrusive achieved force on each of the first subset of teeth. The first set of teeth may be the odd-numbered teeth of the patient's dentition. In some embodiments, the first set of teeth may be every other tooth in patient's dentition. The intrusive movement may be a movement of between 0.05 and 2 mm, such as 0.25 mm.

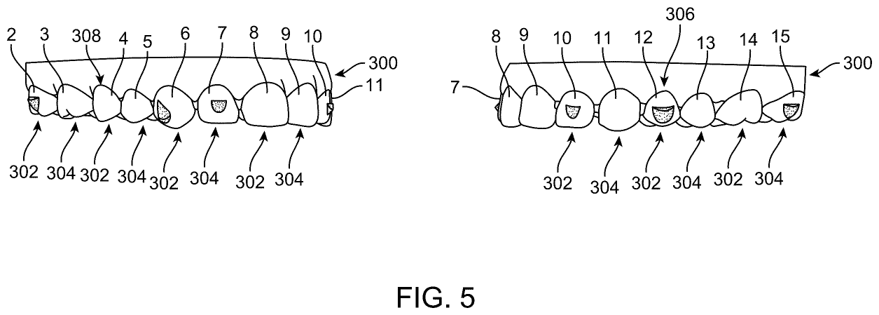

FIG. 5 depicts an actual arch 300 and generated for a stage of treatment in the treatment plan. The first set of teeth may be teeth 302. Simulating the intrusive movement on the patient's teeth may include determining the intrusive force (Fz) imparted on each tooth by an orthodontic appliance shaped to impart the intrusive movement on the patient's teeth. The simulation may be carried out using finite element analysis or other types of digital or numerical simulation of an appliance on the actual arch 300.

At block 144, an intrusive movement is simulated on a second subset of teeth 304 on the actual arch to determine the intrusive achieved force on each of the second subset of teeth. The second set of teeth may be the even teeth of the patient's dentition. In some embodiments, the second set of teeth or every other tooth in patient's dentition between each of the teeth in the first set of teeth. Intrusive movement may be a movement of between 0.05 and 2 mm, such as 0.25 mm.

FIG. 5 depicts an actual arch 300. The second set of teeth may be teeth 304. Simulating the intrusive movement on the patient's teeth may include determining the intrusive force (Fz) imparted on each tooth by an orthodontic appliance shaped to impart the intrusive movement on the patient's teeth. The simulation may be carried out using finite element analysis or other types of digital or numerical simulation of an appliance on the actual arch 300.

At block 146 the intrusive force for each tooth may be normalized. In some embodiments, normalization of the intrusive force may be determined based on the overall intrusive forces applied to the set of teeth. For example when modeling the intrusion on the first set of teeth, the intrusive force on each of the first set of teeth may be determined and then added together to determine the overall intrusive force on the first set of teeth. The normalized force on each of the first set of teeth may then be the force on a particular tooth of the first set of teeth divided by the total force on the first set of teeth.

In some embodiments, the intrusive planned movement when for modeling the retention on the actual arch is added to the original planned movement, such as determined by the treatment plan, for the teeth on the arch. In such an embodiment, the retention of an aligner on the planned arch is modeled using a combination or sum of the actual planned movements and the intrusive movements added to the respective first set and second set of teeth.

In some embodiments, the normalized force on each of the second set of teeth may be the force on a particular tooth of the second set of teeth divided by the total intrusive force on the second set of teeth.

In some embodiments, the normalized retention for the planned arch may be calculated by first modeling the intrusion forces applied by an aligner on the actual arch without the intrusion on the first and second set of teeth or with only the movements from the treatment plan by the aligner and then by modeling the intrusive forces applied by the aligner on the actual arch with both the intrusion on the first and second set of teeth and the tooth movements from the treatment plan.

In some embodiments, the net intrusion force for the planned arch may be the intrusion force modeled by the planned tooth movements subtracted from the intrusion force modeled by the intrusive planned movement determined on the first and second set of teeth and the treatment planned movements. These forces then can be normalized as discussed above. The normalization may occur for both the total intrusion forces applied by the first set of teeth and the second set of teeth and for each of the individual teeth.

FIG. 6 depicts a table that shows the tooth identification (TID), the intrusive force (Fz) applied to each tooth, and the normalized retention for each tooth of the actual arch along with total intrusion force on the even-numbered teeth (the first set of teeth), and the total intrusion force on the odd-numbered teeth (the second set of teeth).

At block 150 the difference between the normalized intrusion forces of the retentive arch are compared to the normalized intrusive forces of the actual or planned arch. FIG. 6 depicts a retention residual in the rightmost column. The value represents the normalized retention of the planned arch subtracted from the normalized retention of the retentive arch. For example, tooth number two has a normalized retention of 0.01 for the planned arch and a normalized retention of 0.04 for the retentive arch. Subtracting the normalized retention of the planned arch from the normalized retention of the retentive arch results in a residual of 0.03.

At block 160 the treatment plan may be modified based on a comparison of the retentive arch with the actual or planned arch. In some embodiments, an aligner may be modified to increase retention by adjusting offset and radius of curvature of the trim line of an orthodontic aligner.

A trim line of an orthodontic aligner for a thermoformed aligner is the gingival edge or the gingival most edge of the orthodontic aligner where the aligner is cut from the thermoformed sheet from which it is formed. The cut is usually where the gingival edge of the crown of the tooth meets the gingiva. Work in relation to this disclosure has shown that extending the cut line beyond the gingival edge and increasing the minimum radius of curvature of the cut line increases the retention of the aligner on the patient's teeth during treatment.

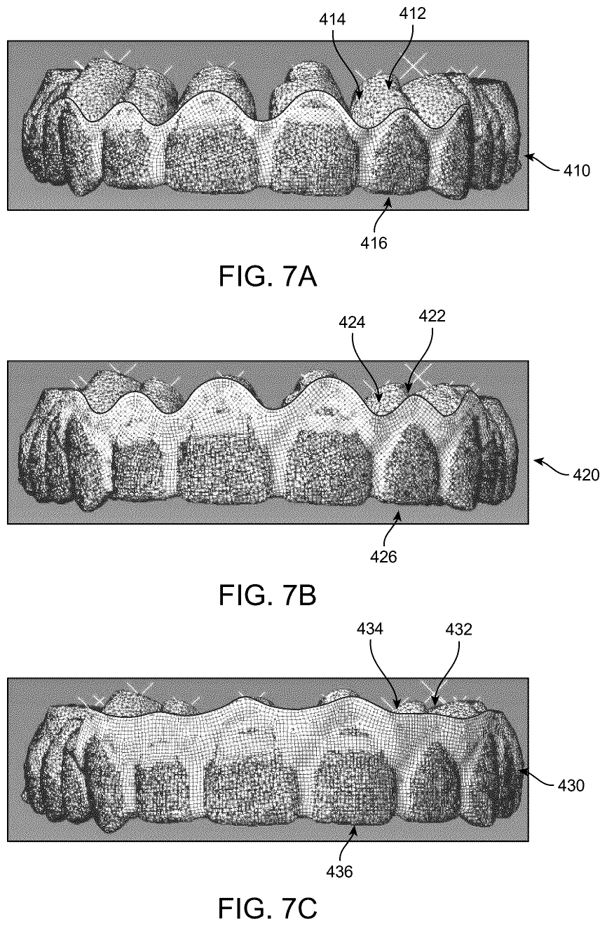

FIGS. 7A-7C depict various changes to a cut line to increase retention on the aligner. In some embodiments, the cut line and radius of curvature may be increased for the teeth having the lowest retention residual, as determined above, and to the teeth immediately adjacent on either side of the tooth with the lowest retention residual. For example, in FIG. 6, teeth 4 and 12 have the highest retention residual. Tooth 308 and tooth 306 in FIG. 5 are teeth 4 and 12. In some embodiments, the cut line may be modified at the tooth receiving cavities of teeth 3, 4, and 5 to increase the retention at tooth 4 and at teeth 11, 12, and 13 to increase the retention at tooth 12.

Referring back to FIGS. 7A-7C, FIG. 7A depicts an aligner having no change in the cut line offset 412, the cut being at the gingival line, and with a radius of curvature 414 of 2 mm. FIG. 7B depicts an aligner having an increase in the line offset 422 of 1.5 mm beyond the gingival line and with a radius of curvature 424 of 2 mm. FIG. 7C depicts an aligner having an increase in the line offset 432 of 1.5 mm beyond the gingival line and with a radius of curvature 434 of 100 mm.

While specific radius of curvature and offset are shown and described herein, other offsets and radius of curvature may be used, the offset may be greater than 1.0 mm and the radius of curvature may be greater than 10 mm. In some embodiments, the offset may be between 1 mm and 2 mm and the radius of curvature may be between 50 mm and 150 mm.

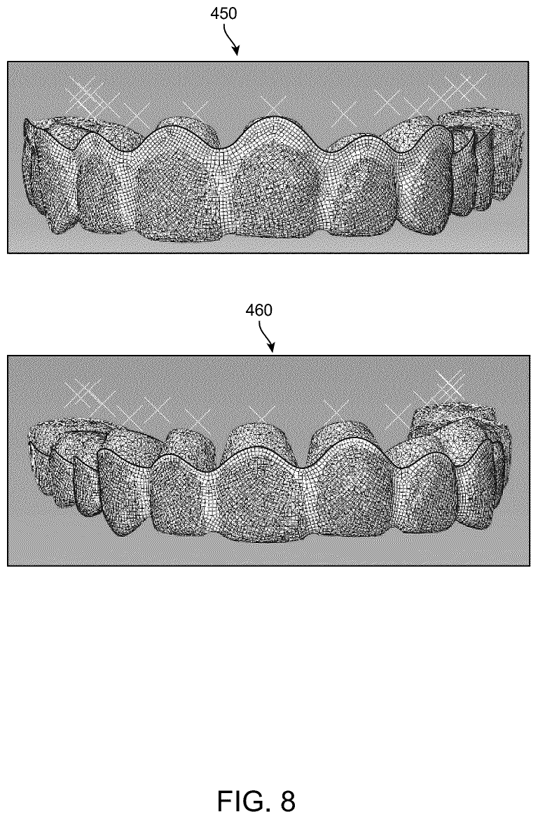

FIG. 8 depicts experimental simulated results of aligner retention as compared to modifications to the cut line of an aligner. Aligner 450 was modified with a 1.5 mm and a 2 mm radius of curvature. Aligner 460 has a standard cutline. Not shown, another aligner may have a 1.5 mm longer cut line from tooth 5 to tooth 12. The results show that the modified aligners imparted more than 15% greater intrusion force on tooth 9 than the unmodified aligner.

Aligners may be modified in other ways. For example, work in relation to this disclosure has shown that by changing the offset of the aligner with respect to the shape of the tooth change the retention of the tooth. The modification of the offset of the aligner with respect to the shape of a tooth for a stage of treatment may increase the force applied by the aligner to the tooth. This increase in applied force increases the retention of the aligner on the tooth through increases in normal forces (e.g., the forces applied to the tooth by the aligner through the deviation of the shape and position of the tooth receiving cavity and the tooth and frictional forces between the aligner (e.g., the forces between the aligner and the tooth that resists movement of the aligner with respect to the tooth, such as sliding of the aligner across the tooth surface).

Frictional forces are forces that resist the relative motion between two surfaces in contact. Friction forces arise due to the interactions between the surfaces at a microscopic level, including mechanical interlocking of surface asperities (irregularities) and adhesive forces where materials tend to stick the materials of the two surfaces together. The surfaces of even seemingly smooth objects, such as an orthodontic aligner and a tooth are rough. They may have tiny peaks and valleys, known as asperities, where the actual contact points between the aligner and tooth surfaces. When the aligner and tooth surfaces come into contact, these asperities interlock. There's also an adhesive component to friction. The adhesive arises because the molecules at the contact surfaces attract each other. The strength of these adhesive forces depends on the nature of the materials in contact and their surface treatments.

Friction may be divided into two different types, static friction and kinetic friction. Static friction is the friction between two surfaces that are not moving relative to each other and is defined by a coefficient of static friction and the normal force between the two surfaces. Kinetic friction is the friction between two surfaces that are moving relative to each other and is defined by a coefficient of kinetic friction and the normal force between the two surfaces. For friction between aligners and teeth, the two surfaces are usually in a static relationship wherein they are not sliding relative to each other, accordingly, it is the static coefficient of friction and the resulting static frictional forces that will be discussed herein, although similar principles apply with respect to the kinetic friction and the resulting frictional forces.

The coefficients of friction between two surfaces may change based on the surface conditions, such as lubrication and other factors. The humidity and moisture in the mouth and the relative smoothness of the teeth and aligner surfaces lead to a relative low coefficient of friction in the mouth.

The equation for friction between an aligner and a tooth is:

$$F = μ \times N$$

Wherein N is the normal force applied between the contacting surfaces of the aligner and the tooth, p is the coefficient of friction between the contacting surfaces of the aligner and the tooth, and F is the resulting frictional force between the contacting surfaces of the aligner and the tooth. Based on this, we can see that as the normal force increases, the increased forces push the surfaces together more firmly. Without being limited by theory, the increased normal force causes more asperities to come into contact, and existing contact points are compressed further, enhancing mechanical interlocking and increasing the frictional forces. In addition, although the apparent contact area between an aligner and tooth surface remains the same (e.g., the surface area of contact between the aligner and the tooth are the same on a macro level, the real area (e.g., at the micro level) of contact increases as the normal force increases because more asperities come into contact, and this effectively increases the surface area where adhesion can occur.

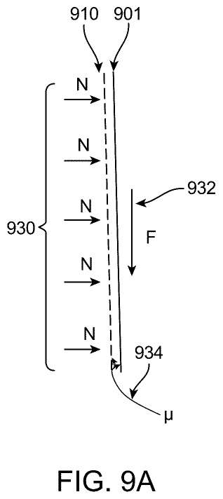

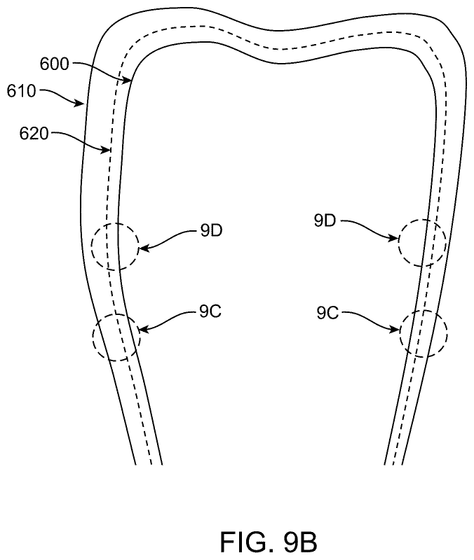

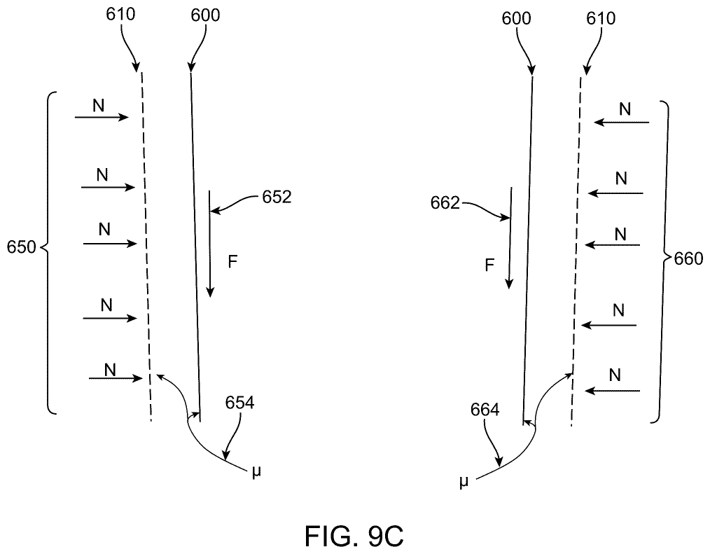

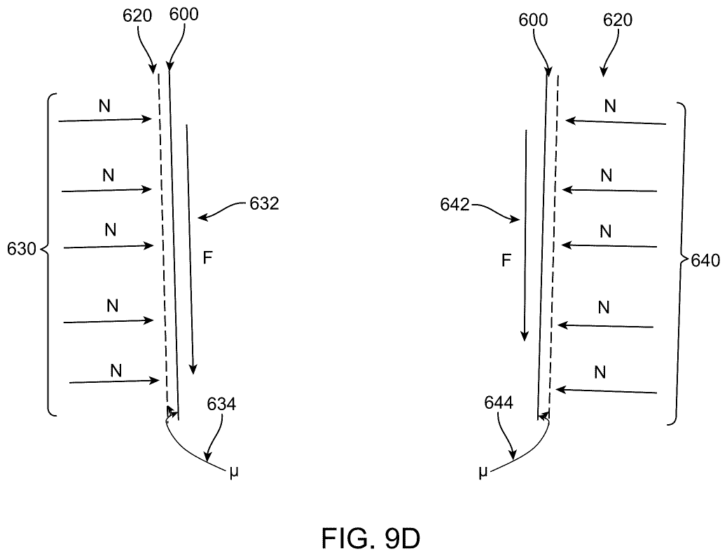

FIG. 9A depicts a diagram of the of the surface of the aligner 901 applying an initial planned normal force 930 to the tooth with the resulting friction 932, such as may be imparted using an aligner before the application of the modifications of FIG. 1. The aligner surface 910 may be pressed against the surface of the tooth 901 with a normal force 930, which may be a distributed load, as represented by the arrows, N. The coefficient of friction 934, between the surfaces of the aligner 910 and the tooth 901, p may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

FIG. 9B shows the outline of a tooth 600 along with two different aligners with tooth receiving cavities having offsets from the shape of the tooth. For example, aligner 610 has a nominal offset from the surface of the tooth. The nominal offset may be a typical offset of an aligner with respect to the shape of the patient's dentition which may allow for retention of the aligner on the tooth along application of tooth movements forces, such as 0.04 mm, and therefore the tooth receiving cavity is larger than the shape of the tooth. In some embodiments, the nominal offset, while well suited for many types of tooth movements, may not be adequate for some tooth movement. Accordingly, some parts of an aligner, such as some tooth receiving cavities and interproximal regions (e.g., the regions between immediately adjacent teeth), may have an offset that is less than nominal or relative to the nominal separation between the tooth and the aligner. For example, the tooth receiving cavity 620 −0.02 mm as compared to the nominal offset, and therefore the tooth receiving cavity is slightly smaller than the shape of a nominal tooth receiving cavity and has an offset from the tooth of 0.02 mm. Decreasing the offset or modifying the aligner to have a negative offset from the nominal offset has shown to increase retention of the aligner.

FIG. 9C depicts diagrams of the surface of the aligner's tooth receiving cavity 610 applying normal forces to the tooth before modifying the tooth receiving cavity shape, such as when the tooth receiving cavity has a nominal offset from the tooth, as discussed herein. The aligner surface 610 may be pressed against the buccal and lingual surfaces (and interproximal surfaces, and in some embodiments, additionally the occlusal surfaces) of the tooth 600. On the buccal side, the aligner 610 presses against the tooth with a normal force 650, which may be a distributed load, as represented by the arrows, N. The coefficient of friction 654, between the surfaces of the aligner 610 and the tooth 600, p, may take into account the surface properties of the aligner and the tooth, environmental factors, such as saliva between the surfaces, etc. The resulting force 652, F, is represented as the frictional force acting in a gingival direction, against the removal of the aligner from the tooth. Such frictional force aids in the retention of the aligner on the tooth and this increases the retention of the aligner on the tooth.

On the lingual side, the aligner 610 presses against the tooth with a normal force 660, which may be a distributed load, as represented by the arrows, N. The coefficient of friction 664, between the surfaces of the aligner 610 and the tooth 600, p may take into account the surface properties of the aligner and the tooth, environmental factors, such as saliva between the surfaces, etc. The resulting force 662, F, is represented as the frictional force acting in a gingival direction, against the removal of the aligner from the tooth. Such frictional force aids in the retention of the aligner on the tooth and this increases the retention of the aligner on the tooth.

Before modifying the shape of the tooth receiving cavity, such as through a modification of the aligner offset from the tooth, such as described herein, the expected retention forces on the aligner from the friction force F, may be lower than those imparted before modification, such that the aligner is not well retained on the teeth.

FIG. 9D depicts the forces that may be applied between a tooth and an aligner with an offset, such as a −0.02 mm offset from nominal, such as aligner 620. FIG. 9D depicts, in more detail, the interaction of the buccal and lingual sides of the tooth receiving cavity and the buccal and lingual sides of the tooth and the associated increase in frictional forces caused by the modification in the aligner.

FIG. 9D depicts diagrams of the of the surface of the aligner's tooth receiving cavity 620 applying a normal force 630 to the tooth by the modifying the tooth receiving cavity shape with an offset created, as discussed herein, with the resulting friction 632, such as may be imparted using an aligner after the application of the modifications of FIG. 1. The aligner surface 620 may be pressed against the buccal and lingual surfaces (and interproximal surfaces, and in some embodiments, additionally the occlusal surfaces) of the tooth 600. On the buccal side, the aligner 620 presses against the tooth with a normal force 630, which may be a distributed load, as represented by the arrows, N. The coefficient of friction 634, between the surfaces of the aligner 620 and the tooth 600, p may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

On the lingual side, the aligner 620 presses against the tooth with a normal force 640, which may be a distributed load, as represented by the arrows, N. The coefficient of friction 644, between the surfaces of the aligner 620 and the tooth 600, p may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

After modifying the shape of the tooth receiving cavity, such as through a modification of the aligner offset from the tooth, such as described herein, the expected retention forces on the aligner from the friction force F, may be high or higher than those imparted before modification, such that the aligner is more well retained on the teeth.

Increased retention of an orthodontic aligner refers to how securely the aligner fits over the teeth, and this has a direct impact on the effectiveness of the aligner in imparting movement forces to the teeth. When an aligner fits snugly and is well retained on the teeth, it is better able to transfer the designed forces to the teeth and may be able to impart greater tooth movement forces on the teeth. High retention also aids in the aligner's contact with the intended surfaces of the teeth, allowing for precise control over the tooth movement. This is particularly useful for complex movements like rotations and extrusions. Although the retention is increased to nonmoving teeth, the increased retention allows for more predictable and greater forces to be applied to moving teeth.

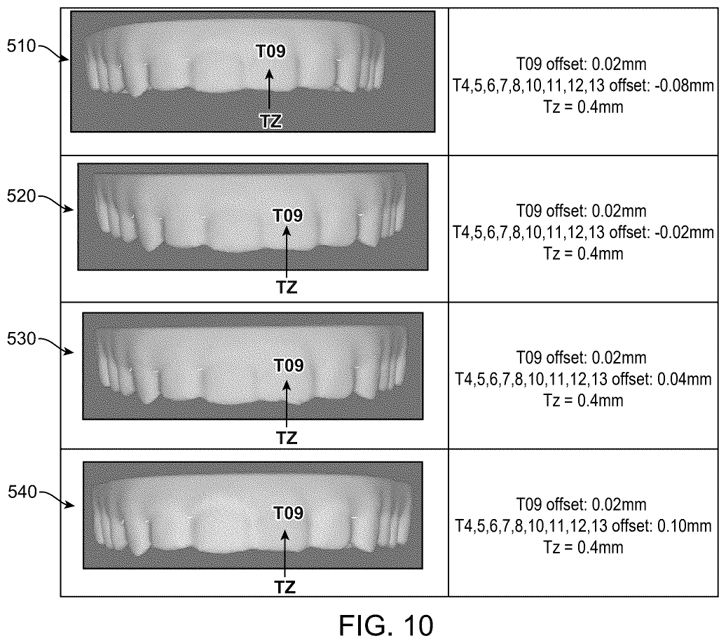

FIG. 10 depicts four different aligners. Aligner 510 has an offset at tooth 9 of 0.02 mm and offset at teeth 4, 5, 6, 7, 8, 10, 11, 12, and 13 of −0.08 mm. Aligner 520 has an offset at tooth 9 of 0.02 mm and offset at teeth 4, 5, 6, 7, 8, 10, 11, 12, and 13 of −0.02 mm. Aligner 530 has an offset at tooth 9 of 0.02 mm and offset at teeth 4, 5, 6, 7, 8, 10, 11, 12, and 13 of 0.04 mm. Aligner 540 has an offset at tooth 9 of 0.02 mm and offset at teeth 4, 5, 6, 7, 8, 10, 11, 12, and 13 of 0.10 mm.

Each aligner was modeled on a patient's dentition with an intrusive movement of 0.04 mm applied to tooth 9. The intrusive force with for aligners with negative offsets to the teeth surrounding tooth 9 is greater than the intrusive force for aligners with positive offsets to the teeth surrounding tooth 9. The increased intrusion force varied from between a greater than 35% increase for the 0.04 mm offset, to greater than 120% increase for the −0.02 mm offset, to greater than 140% increase for the −0.08 mm offset.

In some embodiments, an offset may be applied to tooth receiving cavities of an aligner to the teeth adjacent to immediately adjacent a tooth with high residual retention. In some embodiments, the offset may be applied to tooth receiving cavities of an aligner that are not importing in intrusive movement on to the respective tooth.

Returning to FIG. 1, at block 160 the treatment plan may be modified by changing and offset of the aligner at tooth receiving cavities adjacent to the tooth with low residual retention and/or by increasing the cut line offset and radius of curvature to the tooth with the high residual retention and the teeth immediately adjacent the tooth with a high residual retention.

After modifying the aligner, the process may return to block 140 to reevaluate the retention of the modified aligner and then to block 150 and 160 to generate a retention difference and a further modified aligner, respectively.

The steps at blocks 140, 150, 160 may repeat until adequate retention is achieved. In some embodiments, the steps at blocks 140, 150, 160 may repeat up to three times to increase the retention of the arch. In some embodiments, each iteration the offset may be adjusted at one or more teeth, the negative offset may be increased again (to a more negative value) for teeth where the negative offset was increased (for a more negative value) in a previous iteration. In some embodiments, the cut line offset may be increased again for a tooth where the cut line offset was increased in a previous iteration.

In some embodiments, the offsets and radius of curvature may be added to teeth in a subsequent iteration where such modifications were not made in a previous iteration.

In some embodiments modifications may be made to tooth receiving cavities immediately adjacent a first one or more teeth during a first iteration and modifications may be made to a second one or more teeth, different than the first one or more teeth, during a second subsequent iteration. In some embodiments, modifications may be made to a third one or more teeth, during a third subsequent iteration.

In some embodiments, steps 130, 140, 150, 160 may be repeated for one or more stages of a treatment plan. In some embodiments, the steps 130, 140, 150, 160 may be repeated for every stage of a treatment plan.

The treatment plan may be modified based on the modified aligners generated for the stages of treatment at block 160. After the modifications are complete, the treatment plan and/or the aligner geometries generated using the method 100 may be output such as, as an STL file for fabrication. At block 170 in some embodiments the aligner geometries may be output for direct fabrication, such as for 3D printing including SLA processes. In some embodiments, geometries of molds for fabricating an aligner having the desired shape may be output for fabrication, such as for use in thermoforming.

In some embodiments, one or more aligners for the treatment plan may be fabricated using the geometries. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages.

The various embodiments of the orthodontic appliances presented herein can be fabricated in a wide variety of ways. In some embodiments the aligner may be directly fabricated, such as for 3D printing including SLA processes. In some embodiments, the orthodontic appliances herein (or portions thereof) can be produced using direct fabrication, such as additive manufacturing techniques (also referred to herein as “3D printing) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliances herein can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances herein can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances herein. In some embodiments, material jetting involves jetting or extruding one or more materials onto a build surface in order to form successive layers of the object geometry.

In some embodiments, molds may be fabricated based on the arrangement of teeth in the treatment plan and an aligner having the desired shape may be fabricated using the model, such as via thermoforming. Thermoforming is a manufacturing process used to create aligners from sheets of thermoplastic material. In some embodiments, the mold for the aligner may be directly fabricated. Then, a sheet of thermoplastic material may be heated until the sheet becomes soft and pliable. This may be accomplished using radiant, convection, or contact heating methods. Once the sheet reaches a desired temperature, it is transferred to a forming station where it is molded into shape using an aligner mold. The sheet may be formed into an aligner by placing the sheet over a mold and applying a vacuum underneath the sheet and mold, sucking the plastic down into the mold. In some embodiments, air pressure may applied from above the heated sheet to form the sheet onto the mold. After the thermoplastic has been formed into an aligner, it may be cooled to harden and maintain its form. Finally, excess thermoplastic material may be trimmed from the aligner shape and any other finishing steps may be performed, such as cutting, sanding, and/or polishing to form additional features in the aligner and/or to remove rough edges.

As shown in FIG. 11, an embodiment of a method 1100 for designing a retentive dental appliance may include receiving a 3D model of the state of the oral cavity at block 1110, generating a treatment plan for the patient at block 1120, determining stationary teeth for one or more stages of the treatment plan at block 1130, modifying movement of a stationary tooth for a state of the treatment plan at block 1140, outputting a revised treatment plan at block 1150, and fabricating an appliance for the stage of the treatment plan at block 1160.

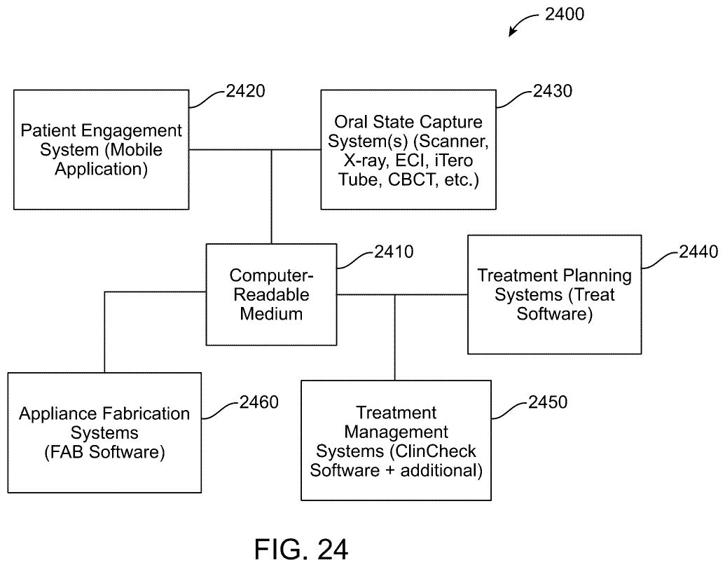

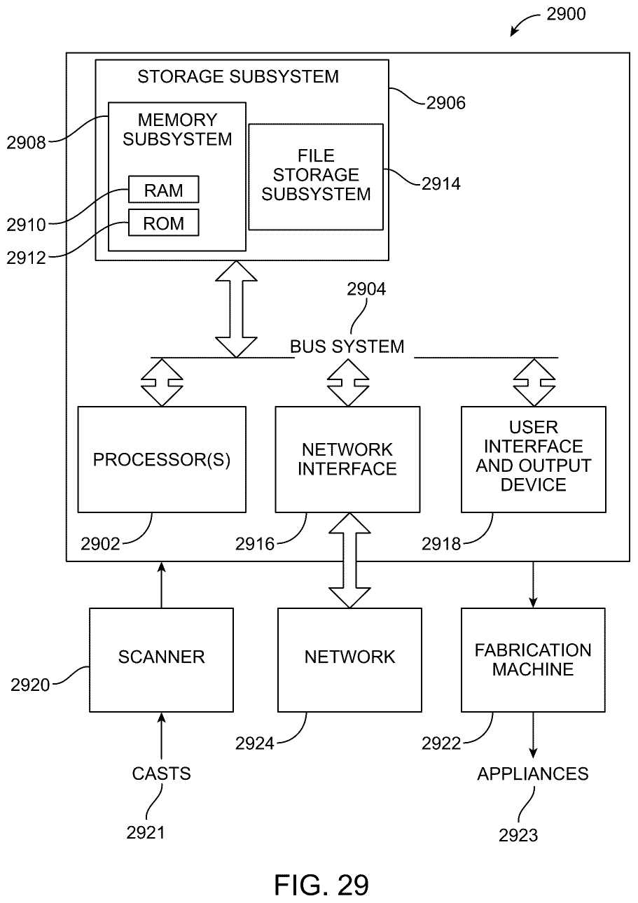

The process shown in FIG. 11, and the other processes described herein, may be performed by any suitable computer-executable code and/or computing system, including the system(s) illustrated in FIGS. 24 and 29. In one example, each of the steps of the process 1100 shown in FIG. 11 may represent an algorithm whose structure includes and/or is represented by multiple sub-steps, examples of which will be provided in greater detail below.

At block 1110 a 3D model of the state of the patient's oral cavity may be received. The 3D model may be a digital representation and may include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.). The 3D model of the state of the patient's oral cavity may also include subsurface data of the patient's intraoral cavity such as information related to the size, shape, location, and orientation of the roots of the patient's teeth and features of the patient's dental tissues such as the interior of the patient's teeth, the patient's jaws, and the patient's gingiva. Surface typology and subsurface information may be provided by x-rays, CBCT scans, and two-dimensional photos of the patient's dentition and oral cavity.

In some embodiments, at block 1110, the patient's oral cavity may be imaged using an intraoral scanner, an x-ray scanner, a CBCT scanner, or other imaging systems such as infrared and visible light images which may be 2D images.

In some embodiments, the patient's dentition may be segmented at block 1110. Tooth segmentation of dental scans analyzes and processes images obtained through various dental scanning techniques (such as X-rays, CBCT scans, CT scans, or 3D imaging) to segment teeth and other dental tissues, such as gingiva, to accurately distinguish and separate the different teeth and/or components of the teeth and intraoral tissue from the surrounding tissue and from each other in these digital images. This separation may be used for diagnosis, treatment planning, orthodontic study, and the creation of dental prosthetics.

At block 1120 a treatment plan for moving a patient's teeth from an initial arrangement towards a final arrangement is generated. The patient's initial arrangement may be based on the state of the patient's oral cavity determined at block 1110. In some embodiments, at block 1120 a final or target arrangement may be determined. The final or target arrangement may be the goal for an arrangement of the patient's teeth at the end of treatment.

A treatment plan may include multiple stages for moving the patient's teeth from an initial arrangement to towards the final arrangement. The treatment stages can be incremental repositioning stages of an orthodontic treatment procedure designed to move one or more of the patient's teeth from an initial tooth arrangement towards a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, and/or any other suitable criteria.

For each stage of treatment a digital model of the patient's dentition may be generated and used as a basis for forming an appliance such as an orthodontic aligner for that stage of treatment. In some embodiments, treatment planning may also include determining the location, shape, and orientation of other appliance features such as attachments on the patient's teeth, and other aligner features described herein.

At block 1130 the method may include determining which teeth for a stage of a treatment plan are stationary during the stage. In some embodiments, at block 1130 the process may include determining which teeth are stationary at one or more stages of a treatment plan. In some embodiments, at block 1130 the process may include determining which teeth are stationary at each stage of a treatment plan. In some embodiments, at block 1130 the process may include determining which teeth are stationary at every stage of a treatment plan.

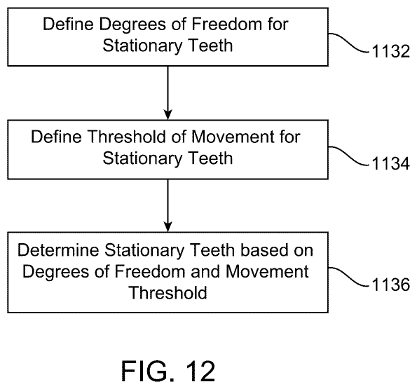

FIG. 12 depicts aspects of a method for determining which teeth are stationary teeth for a stage of treatment and may be used to determine whether a tooth is stationary for one or more, such as all, of the stages of treatment. The process may include defining degrees of freedom for stationary teeth at block 1132, defining a threshold of movement for stationary teeth at block 1134, and determining which teeth are stationary based on the degrees of freedom and the movement threshold for one or more stages of treatment.

At block 1132 the process of defining the degrees of freedom for stationary teeth may include defining the degrees of freedom according to directions in the mouth. For example, in some embodiments, the degrees of freedom may be translational degrees of freedom along a buccal-lingual axis, a mesial-distal axis, and/or a gingival-occlusal axis. In some embodiments, the degrees of freedom may include rotational degrees of freedom about the buccal-lingual axis, the mesial distal axis, and/or the gingival-occlusal axis.

In some embodiments, the degrees of freedom may be translational degrees of freedom along the direction of movement of a tooth.

In some embodiments, the degrees of freedom may include rotational degrees of freedom about the buccal-lingual axis, the mesial distal axis, and/or the gingival-occlusal axis. In some embodiments, the degrees of freedom may include rotational degrees of freedom about an axis of rotation of a tooth.

In some embodiments, the translational degrees of freedom may be three translational degrees of freedom, each along one of three orthogonal axis.

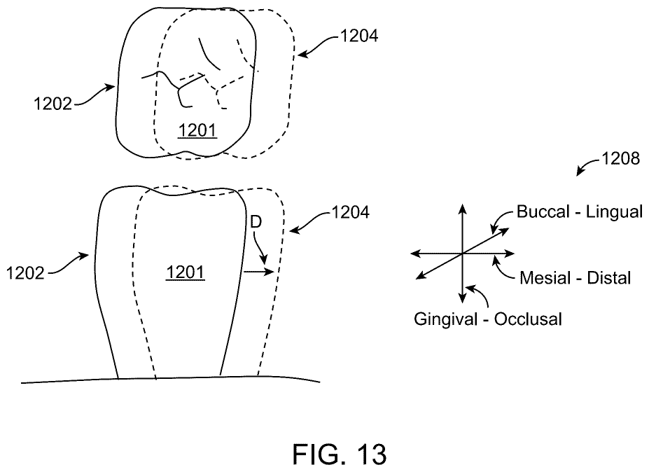

FIG. 13 depicts an example of degrees of freedom 1208. Although the degrees of freedom 1208 are along a buccal-lingual axis, a mesial-distal axis, and/or a gingival-occlusal axis, degrees of freedom may represent other degrees of freedom discussed herein.

Referring back to FIG. 12, at block 1134 the threshold for movement may be defined. A tooth movement below the threshold may be considered a stationary tooth and/or above which a tooth may be considered a moving tooth. In some embodiments, a tooth movement below the threshold may be considered to be a non-moving tooth and/or above which a tooth may be considered a non-stationary tooth.

In some embodiments, the threshold for translational movement may be between about 0 mm and about 0.10 mm of movement, for a single stage of treatment. In some embodiments, the threshold for movement may be set at less than 0.10 mm, such as, less than 0.08 mm, 0.06 mm, 0.05 mm, 0.04 mm, or 0.02 mm. Movement above the movement threshold indicates that the tooth is considered a moving tooth and movement for a stage of treatment below the threshold may indicate that the tooth is not moving.

In some embodiments, the threshold for rotational movement may be between 0 degrees and 10 degrees, for a single stage of treatment. In some embodiments, the threshold for movement may be less than 10 degrees, such as less than 9 degrees, less than 8 degrees, less than 7 degrees, less than 6 degrees, less than 5 degrees, less than 4 degrees, or less than 2 degrees. Movement above the movement threshold indicates that the tooth is considered a moving tooth and movement for a stage of treatment below the threshold may indicate that the tooth is not moving.

In some embodiments, the threshold may be set in advance, such as before receiving a treatment plan. In some embodiments, the threshold may be set individually for a treatment plan. In some embodiments, a first threshold may be set for a first stage of a treatment plan and a second threshold may be set for a second stage of the treatment plan. In some embodiments, a first threshold may be set for a first degree of freedom, a second threshold may be set of a second degree of freedom, and a third threshold may be set of a third degree of freedom. In some embodiments, a threshold may be set for each degree of freedom. In some embodiments, a single threshold may be set and applied to each degree of freedom, such as a translational movement threshold for translational degrees of freedom and a rotational threshold for rotational decrees of freedom.

At block 1136 stationary and/or moving teeth may be determined based on the degree of freedom and the movement threshold. FIG. 13 depicts a tooth 1201 from a buccal side view and an occlusal top view. In FIG. 13, the tooth 1201 is moving a distance D in a mesial-distal direction from a first position 1202 to a second position 1204 during a stage of treatment, while moving no distance in the buccal-lingual and gingival-occlusal degrees of freedom. If the threshold for movement is less than the distance D, but greater than 0 mm, then the tooth may be considered stationary in the buccal-lingual and gingival-occlusal degrees of freedom and moving or non-stationary in the mesial-distal degree of freedom.

In some embodiments, the degree or degrees of freedom may be based on the direction of tooth movement. For example, in some embodiments, a tooth may move diagonally, such as in both the mesial-distal degree of freedom and in the buccal-lingual degree of freedom. In some embodiments, a first degree of freedom may be defined in the direction of tooth displacement or movement during a stage of treatment. In some embodiments, orthogonal degrees of freedom may be defined relative to the direction or tooth displacement or movement.

In some embodiments, if the tooth is moving in the direction of movement more than the threshold, the tooth may be considered a moving tooth. If the tooth is moving in the direction of movement less than the threshold then the tooth may be considered stationary. In such embodiments, the tooth does not move in the orthogonal degrees of freedom, because those degrees of freedom are orthogonal to the movement.

The stationary teeth and/or moving teeth may be determined for more than one stage of treatment. The stationary teeth and/or moving teeth may be determined for one or more stages of treatment. The stationary teeth and/or moving teeth may be determined for a first stage of treatment.

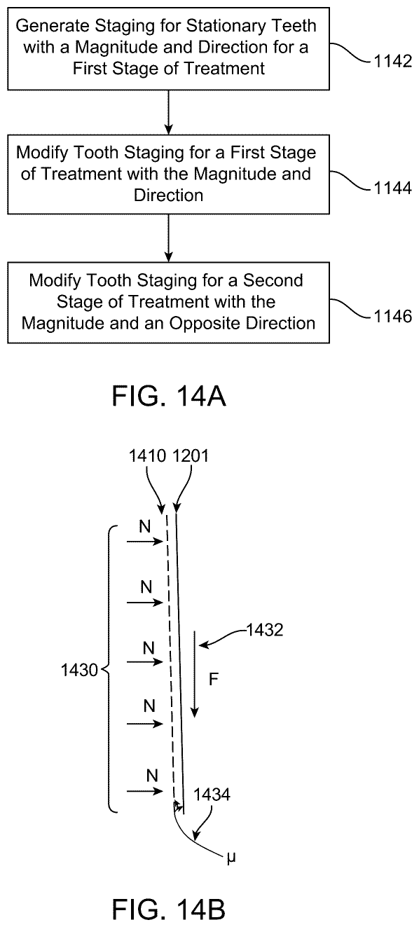

Returning to FIG. 11, the process may proceed to block 1140. At block 1140, the movement of a stationary tooth may be modified for one or more stages of treatment. FIG. 14 shows the process for modifying the movement of a stationary tooth. At block 1142, a first staging for a stationary tooth may be generated to move by a first magnitude and direction. The modification may add the magnitude and the direction of the modification to the tooth's initially planned movement. For example, if a tooth is stationary because it is determined to be moving a distance D that is less than a distance threshold, then the modified tooth movement for the stage may be the distance D plus the magnitude and direction of the additional movement. If a tooth is stationary because it is determined to be moving 0 mm in a degree of freedom that is less than a distance threshold, then the modified tooth movement for the stage may be the magnitude and direction of the additional movement.

In some embodiments, the generated staging may have a change in magnitude of between 0.01 and 0.05 mm in a first direction along a stationary degree of freedom.

The modification of the movement of a tooth for a stage of treatment may increase the force applied by the aligner to the tooth. This increase in applied force can increase the retention of the aligner on the tooth through increases in normal forces (e.g., the forces applied to the tooth by the aligner through the deviation of the shape and position of the tooth receiving cavity and the tooth) and frictional forces between the aligner and the tooth (e.g., the forces between the aligner and the tooth that resists movement of the aligner with respect to the tooth, such as sliding of the aligner across the tooth surface).

Frictional forces are forces that resist the relative motion between two surfaces in contact. Friction forces arise due to the interactions between the surfaces at a microscopic level, including mechanical interlocking of surface asperities (irregularities) and adhesive forces where materials tend to stick the materials of the two surfaces together. The surfaces of even seemingly smooth objects, such as an orthodontic aligner and a tooth, are rough at this level. They may have tiny peaks and valleys, known as asperities, where the actual contact between the aligner and tooth surfaces occur. When the aligner and tooth surfaces come into contact, these asperities interlock. There's also an adhesive component to friction. The adhesive component arises because the molecules at the contact surfaces attract each other. The strength of these adhesive forces depends on the nature of the materials in contact and their surface treatments.

FIG. 14B depicts a diagram of the of the surface of the aligner 1410 applying an initial planned normal force 1430 to the tooth with the resulting friction 1432, such as may be imparted using an aligner before the application of the modifications of FIG. 14. The aligner surface 1410 may be pressed against the surface of the tooth 1201 with a normal force 1430, which may be a distributed load, as represented by the arrows, N. The coefficient of friction 1434, between the surfaces of the aligner 1410 and the tooth 1201, p may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

Before modifying the staging of a tooth, such as described herein by implementing a deviation of the aligner tooth receiving cavity, the expected retention forces on the aligner from the friction force F, may be low, such that the aligner is not well retained on the teeth.

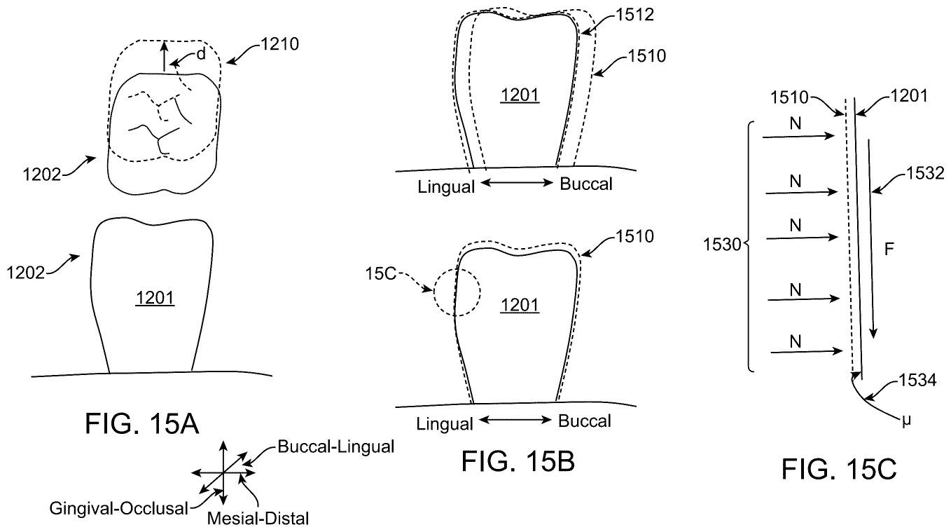

At block 1144, the staging for a tooth for a first stage of treatment may be modified. Although described as a first stage of treatment, the first stage discussed here may not be the first in time stage of a treatment plan and may be a second or other intermediate stage of a treatment plan. FIG. 15A depicts a modified staging along a stationary degree of freedom. For example, the tooth shown in FIG. 15A is considered to be stationary in buccal-lingual degree of freedom at block 1130. At block 1140 and in particular at block 1144, the tooth 1201 may modified to move in a direction d to a second position 1204 during the first stage of treatment. In the embodiment shown in FIG. 15A, the degree of freedom without movement is along the buccal-lingual axis. As shown, the modified movement in FIG. 15A is along the buccal-lingual axis in the buccal direction.

The upper portion of FIG. 15B depicts the initial relationship between the tooth 1201 and an aligner's tooth receiving cavity position 1512. The initial relationship shows that the aligner may not impart much, if any movement forces on the tooth 1210 because the tooth receiving cavity position 1512 is in the same, or nearly the same, location as the tooth, when the aligner is applied to the tooth.

The tooth receiving cavity position 1510 represents the modified displacement of an aligner tooth receiving cavity with respect to the position of the tooth 1201 after the modification made as described with reference to block 1144 of FIG. 14. The displacement of the tooth receiving cavity in the buccal direction to the position 1510 results in increased forces on the lingual side of the tooth by the aligner when the aligner is worn by the patient because of the deformation of the aligner when it is placed on the tooth pulls on and deforms the lingual side of the tooth receiving cavity.

The bottom of FIG. 15B depicts the tooth receiving cavity on the tooth after the aligner has been placed on the dentition of the patient. As shown in FIG. 15B, the tooth receiving cavity 1510 is physically deformed and displaced to fit over the tooth 1201. In doing so, the lingual side of the tooth receiving cavity is more firmly pressed against the lingual side of the tooth. FIG. 15C depicts, in more detail, the interaction of the lingual side of the tooth receiving cavity and the lingual side of the tooth and the associated increase in frictional forces caused by the modification in the aligner.

FIG. 15C depicts a diagram of the of the surface of the aligner tooth receiving cavity 1510 applying a normal force 1530 to the tooth by the modified tooth receiving cavity position created by the modification at block 1144 with the resulting friction 1532, such as may be imparted using an aligner after the application of the modifications of FIG. 14. The aligner surface 1510 may be pressed against the lingual surface of the tooth 1201 with a normal force 1530, which may be a distributed load, as represented by the arrows, N. The coefficient of friction 1534, between the surfaces of the aligner 1510 and the tooth 1201, p may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

After modifying the staging of a tooth, such as described herein by implementing a deviation of the aligner tooth receiving cavity, the expected retention forces on the aligner from the friction force F, may be high or higher than those imparted before modification, such that the aligner is more well retained on the teeth.

Increased retention of an orthodontic aligner refers to how securely the aligner fits over the teeth, and this has a direct impact on the effectiveness of the aligner in imparting movement forces to the teeth. When an aligner fits snugly and is well retained on the teeth, it is better able to transfer the designed forces to the teeth and may be able to impart greater tooth movement forces on the teeth. High retention also aids in the aligner's contact with the intended surfaces of the teeth, allowing for precise control over the tooth movement. This is particularly useful for complex movements like rotations and extrusions. Although the retention is increased to nonmoving teeth, the increased retention allows for more predictable and greater forces to be applied to moving teeth.

At block 1146, the staging for a tooth for a second stage of treatment, subsequent to the first stage of treatment, may be modified. Although described as a second stage of treatment, the second stage discussed here may not be the second in time stage of a treatment plan and may be a second or other intermediate stage of a treatment plan, such as a stage immediately after the first stage discussed in block 1144 or a subsequent stage. FIG. 16A depicts a modified staging along a stationary degree of freedom. For example, the tooth shown in FIG. 16A is considered to be stationary in buccal-lingual degree of freedom at block 1130. At block 1140 and in particular at block 1146, the tooth 1201 may be modified to move in a direction −d, opposite in magnitude and direction to the movement of a prior stage, to a second position 1212 during the second stage of treatment. In the embodiment shown in FIG. 6A, the degree of freedom without movement is along the buccal-lingual axis. As shown, the modified movement in FIG. 16A is along the buccal-lingual axis in the lingual direction, which is a direction opposite the direction of the displacement in the previous stage.

The upper portion of FIG. 16B depicts the initial relationship between the tooth 1201 and an aligner's tooth receiving cavity position 1612. The initial relationship shows that the aligner may not impart much, if any movement forces on the tooth 1201 because the tooth receiving cavity position 1612 is in the same, or nearly the same, location as the tooth, when the aligner is applied to the tooth.

The tooth receiving cavity position 1610 represents the modified displacement of an aligner tooth receiving cavity with respect to the position of the tooth 1201 after the modification made as described with reference to block 1146 of FIG. 14. The displacement of the tooth receiving cavity in the lingual direction to the position 1610 results in increased forces on the buccal side of the tooth by the aligner when the aligner is worn by the patient because of the deformation of the aligner when it is placed on the tooth pulls on and deforms the buccal side of the tooth receiving cavity.

The bottom of FIG. 16B depicts the tooth receiving cavity on the tooth after the aligner has been placed on the dentition of the patient. As shown in FIG. 16B, the tooth receiving cavity 1610 is physically deformed and displaced to fit over the tooth 1201. In doing so, the buccal side of the tooth receiving cavity is more firmly pressed against the lingual side of the tooth. FIG. 16C depicts, in more detail, the interaction of the buccal side of the tooth receiving cavity and the buccal side of the tooth and the associated increase in frictional forces caused by the modification in the aligner.

FIG. 16C depicts a diagram of the surface of the aligner tooth receiving cavity 1610 applying a normal force 1630 to the tooth by the modified tooth receiving cavity position created by the modification at block 1146 with the resulting friction 1632, such as may be imparted using an aligner after the application of the modifications of FIG. 14. The aligner surface 1610 may be pressed against the buccal surface of the tooth 1201 with a normal force 1630, which may be a distributed load, as represented by the arrows, N. The coefficient of friction 1634, between the surfaces of the aligner 1610 and the tooth 1201, μ, may take into account the surface properties of the aligner and the tooth, environmental factors, such as saliva between the surfaces, etc. The resulting force, F, is represented as the frictional force acting in a gingival direction, against the removal of the aligner from the tooth. Such frictional force aids in the retention of the aligner on the tooth and this increases the retention of the aligner on the tooth.

After modifying the staging of a tooth, such as described herein by implementing a deviation of the aligner tooth receiving cavity, the expected retention forces on the aligner from the friction force F, may be high or higher than those imparted before modification, such that the aligner is more well retained on the teeth.

Increased retention of an orthodontic aligner refers to how securely the aligner fits over the teeth, and this has a direct impact on the effectiveness of the aligner in imparting movement forces to the teeth. When an aligner is well retained on the teeth, it is better able to transfer the designed forces to the teeth and may be able to impart greater tooth movement forces on the teeth. High retention also aids in the aligner's contact with the intended surfaces of the teeth, allowing for precise control over the tooth movement. This is particularly useful for complex movements like rotations and extrusions. Although the retention is increased to nonmoving teeth, the increased retention allows for more predictable and greater forces to be applied to moving teeth.

In some embodiments, the process may repeat blocks 1144 and blocks 1146 modifying the movement one or more stationary teeth a determined magnitude and alternating directions for subsequent stages. In some embodiments, a tooth that is stationary in a first one or more stages may be a non-stationary tooth during one or more subsequent stages. The tooth's position may be modified during the first one or more stages to increase retention but not modified during non-stationary stages. In some embodiments, a non-stationary tooth may not have its movement modified for a first one or more stages and then, during stages when it is stationary, may have its movement modified. In some embodiments, a tooth's position may be modified as described herein, alternating direction at a determined magnitude, during stationary stages and not having its movement modified during non-stationary stages.

FIG. 16D depicts three tables, each showing aspects of the tooth movements and modifications to tooth positions described herein. The top table shows the planned movements of a subset of the patient's teeth according to a treatment plan. For simplicity, the movements shown are only in the buccal-lingual direction, with positive numbers meaning displacement or movement in the buccal direction and negative numbers meaning displacement of movement in the lingual direction. Although only movements of teeth 4 through 8 for stages 1 through 8 are depicted in the table, the treatment plan may include movements for all of the patient's teeth for each stage of treatment, which may be more or fewer stages of treatment than the 8 depicted. As shown in the upper table, tooth 4, the upper right, second bicuspid, is not scheduled to move for any of the eight stages of treatment for a total cumulative movement of 0.0 mm over the eight stages of treatment. Tooth 5, the upper right, first bicuspid, is scheduled to move in 0.15 mm in the buccal direction in stages 4, 5, and 6 with no movement scheduled for the other stages of treatment for a total cumulative movement of 0.45 mm in the buccal direction over the eight stages of treatment. Tooth 6, the upper right cuspid, is scheduled to move 0.05 mm in the lingual direction for stages 1, 2, and 3 with no movement scheduled for the other stages of treatment for a total cumulative movement of 0.15 mm in the lingual direction over the eight stages of treatment. Tooth 7, the upper right lateral incisor is scheduled to move 0.05 mm in the lingual direction for stages 1, 2, and 3 with no movement for the other stages of treatment for a total cumulative movement of 0.15 mm in the buccal direction over the eight stages of treatment. Tooth 8, the upper right, central incisor is scheduled to move 0.20 mm for each of the stages 1, 2, 3, 4, 5, and 6, with no movement for the other stages of treatment for a total cumulative movement of 1.20 mm in the buccal direction over the eight stages of treatment.

The middle table shows the planned deviations of movements of a subset of the patient's teeth according to a treatment plan. The deviations are of a magnitude of 0.03 mm along the buccal lingual direction for non-moving teeth. The threshold for non-moving teeth in this example is 0.06 mm, so teeth whose movements for a stage or treatment less than 0.06 mm in the buccal direction (represented at 0.06 mm) or less than 0.06 mm in the lingual direction (represented at −0.06 mm) are consider non-moving teeth. The planned deviation for this treatment plan 0.03 mm.

For simplicity, the movements shown are only in the buccal-lingual direction, with positive numbers meaning displacement or movement in the buccal direction and negative numbers meaning displacement of movement in the lingual direction. Although only deviations of teeth 4 through 8 for stages 1 through 8 are depicted in the table, the treatment plan may include deviations for all of the patient's teeth for each stage of treatment, which may be more or fewer stages of treatment than the 8 depicted. As shown in the upper table, tooth 4, the upper right, second bicuspid, is considered a non-moving tooth along the buccal-lingual axis not scheduled to move for any of the eight stages of treatment for a total cumulative movement of 0.0 mm over the eight stages of treatment. Tooth 5, the upper right, first bicuspid, is scheduled to move in 0.15 mm in the buccal direction in stages 4, 5, and 6 with no movement scheduled for the other stages of treatment for a total cumulative movement of 0.45 mm in the buccal direction over the eight stages of treatment. Tooth 6, the upper right cuspid, is scheduled to move 0.05 mm in the lingual direction for stages 1, 2, and 3 with no movement scheduled for the other stages of treatment for a total cumulative movement of 0.15 mm in the lingual direction over the eight stages of treatment. Tooth 7, the upper right lateral incisor is scheduled to move 0.05 mm in the lingual direction for stages 1, 2, and 3 with no movement for the other stages of treatment for a total cumulative movement of 0.15 mm in the buccal direction over the eight stages of treatment. Tooth 8, the upper right, central incisor is scheduled to move 0.20 mm for each or stages 1, 2, 3, 4, 5, and 6, with no movement for the other stages of treatment for a total cumulative movement of 1.20 mm in the buccal direction over the eight stages of treatment.

As shown in the upper table, tooth 4, the upper right, second bicuspid, is considered a non-moving tooth along the buccal-lingual because its movements for all eight stages of treatment is 0.0 mm. Accordingly, a deviation is applied to the tooth for each of the eight stages, with the position of the tooth receiving cavity being offset by 0.03 mm in a lingual direction for stages 1, 3, 5, and 7 and being offset of 0.03 mm in the buccal direction for stages 2, 4, 6, and 8. Tooth 5, the upper right, first bicuspid, is considered a moving tooth for stages 4, 5, and 6 because it is scheduled to move in 0.15 mm in the buccal direction for those stages of treatment, which is greater than the 0.06 mm threshold. Accordingly, the movement offset is only applied to the tooth at stages 1, 2, 3, 7, and 8, with the tooth being displaced 0.03 mm in the buccal direction for stages 1, 3, and 8 and 0.03 mm in the lingual direction for stages 2 and 7.

Tooth 6, the upper right cuspid, is considered a non-moving tooth for all 8 stages of treatment because it is scheduled to move 0.05 mm in the lingual direction for stages 1, 2, and 3, which is less than the threshold of 0.06 mm and with no movement scheduled for the other stages of treatment. Accordingly, a deviation is applied to the tooth for each of the eight stages, with the position of the tooth receiving cavity being offset by 0.03 mm in a lingual direction for stages 1, 3, 5, and 7 and being offset of 0.03 mm in the buccal direction for stages 2, 4, 6, and 8. Tooth 7, the upper right lateral incisor is considered a non-moving tooth for all 8 stages of treatment because it is scheduled to move 0.05 mm in the buccal direction for stages 1, 2, and 3, which is less than the threshold of 0.06 mm and with no movement scheduled for the other stages of treatment. Accordingly, a deviation is applied to the tooth for each of the eight stages, with the position of the tooth receiving cavity being offset by 0.03 mm in a buccal direction for stages 1, 3, 5, and 7 and being offset of 0.03 mm in the lingual direction for stages 2, 4, 6, and 8. Tooth 8, the upper right, central incisor is considered a non-moving tooth only for stages 7 and 9 because it is scheduled to move 0.20 mm for each or stages 1, 2, 3, 4, 5, and 6, which is greater than the threshold and with no movement for the other stages of treatment. Accordingly a deviation is only applied at stages 7 and 8 with a deviation of 0.03 mm in lingual direction at stage 7 and 0.03 mm in the buccal direction for stage 8.

The bottom table shows the planned movements of a subset of the patient's teeth according to a treatment plan after the deviations of the middle table have been applied to the planned movements of the upper table. The lower table shows the final tooth movements to be applied during the treatment plan. For simplicity, the movements shown are only in the buccal-lingual direction, with positive numbers meaning displacement or movement in the buccal direction and negative numbers meaning displacement of movement in the lingual direction. Although only movements of teeth 4 through 8 for stages 1 through 8 are depicted in the table, the treatment plan may include movements for all of the patient's teeth for each stage of treatment, which may be more or fewer stages of treatment than the 8 depicted. As shown in the lower table, tooth 4, the upper right, second bicuspid, for stage 1, is now planned to move 0.03 mm in the lingual direction, which is the sum of the planned movement for tooth 4 at stage 1 shown in the upper table and the deviation for tooth 4, stage 1 in the middle table. The cumulative movement remains 0.0 mm because the deviation over the 8 stages cancel each other out over the course of treatment.

Tooth 5, the upper right, first bicuspid, the movements are also added, as discussed herein, however the cumulative movement is 0.48 mm in the buccal direction, which is 0.03 mm greater than the original treatment plan. In some embodiments, the deviation at stage 8, or a final stage of treatment, or during another, later or earlier stage of treatment, may be modified. For example, the deviation at stage 8 or another of the stages of treatment may be removed such that the deviations cancel each other out. In some embodiments, an additional deviation may be added at another stage of treatment so that the deviations cancel each other out. In some embodiments, the deviation may be small enough that no further modifications or deviations are made and the tooth's cumulative movement is changed to 0.48 mm.

The lower table also shows the resulting tooth movements of teeth 6, 7, and 8, based on the addition of the movements and deviations of the corresponding teeth in the upper and middle tables. These tables show that while the planned deviations alternating in each direction for each stage of the treatment plan for the non-moving teeth, the total tooth movements remain the same, or very close to the same, such as within one deviation of the planned movement for the entire treatment. The result of the deviations is greater retention for the stages of treatment, without significantly modifying the end result of the treatment.

At block 1150 the treatment plan may be modified based on the modified aligners generated for the stages of treatment at block 1140. After the modifications are complete, the treatment plan and/or the aligner geometries generated using the method 1100 may be output such as an STL file for fabrication. In some embodiments the aligner geometries may be output for direct fabrication, such as for 3D printing including SLA processes. In some embodiments, geometries of molds for fabricating an aligner having the desired shape may be output for fabrication, such as for use in thermoforming.

At block 1160, in some embodiments, one or more aligners for the treatment plan may be fabricated using the geometries. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages.

The various embodiments of the orthodontic appliances presented herein can be fabricated in a wide variety of ways. In some embodiments the aligner may be directly fabricated, such as for 3D printing including SLA processes. In some embodiments, the orthodontic appliances herein (or portions thereof) can be produced using direct fabrication, such as additive manufacturing techniques (also referred to herein as “3D printing) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances herein. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliances herein can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances herein can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances herein. In some embodiments, material jetting involves jetting or extruding one or more materials onto a build surface in order to form successive layers of the object geometry.

In some embodiments, molds may be fabricated based on the arrangement of teeth in the treatment plan and an aligner having the desired shape may be fabricated using the model, such as via thermoforming. Thermoforming is a manufacturing process used to create aligners from sheets of thermoplastic material. In some embodiments, the mold for the aligner may be directly fabricated. Then, a sheet of thermoplastic material may be heated until the sheet becomes soft and pliable. This may be accomplished using radiant, convection, or contact heating methods. Once the sheet reaches a desired temperature, it is transferred to a forming station where it is molded into shape using an aligner mold. The sheet may be formed into an aligner by placing the sheet over a mold and applying a vacuum underneath the sheet and mold, sucking the plastic down into the mold. In some embodiments, air pressure may applied from above the heated sheet to form the sheet onto the mold. After the thermoplastic has been formed into an aligner, it may be cooled to harden and maintain its form. Finally, excess thermoplastic material may be trimmed from the aligner shape and any other finishing steps may be performed, such as cutting, sanding, and/or polishing to form additional features in the aligner and/or to remove rough edges.

### Topology-Induced Friction

Orthodontic appliance retention is traditionally enhanced by adding composite attachments. While effective, attachments can add clinical overhead and compromise aesthetics. With topography-induced friction, discussed herein, the surface topology of aligners is modified to increase the friction coefficient between the inner surface of an attachment-free aligner and the enamel it contacts.

As discussed herein, Frictional force, F, is the product of the normal force, N, and the coefficient of friction, p. Frictional force can be increased by increasing one or both of the normal force and the coefficient of friction. With topography-induced friction, the frictional force and the associated retention force is increased though an increase in the friction coefficient brought on by changes in the surface topography of the aligner by forming micro-roughness features on buccal, lingual, and/or occlusal contact zones of the appliance's inner surface. Direct-fabrication (e.g., SLA, DLP, FDM, MJF, SLS, and others described herein) and other technologies may be used to generate sub-100 μm surface features to increase static and kinetic friction.

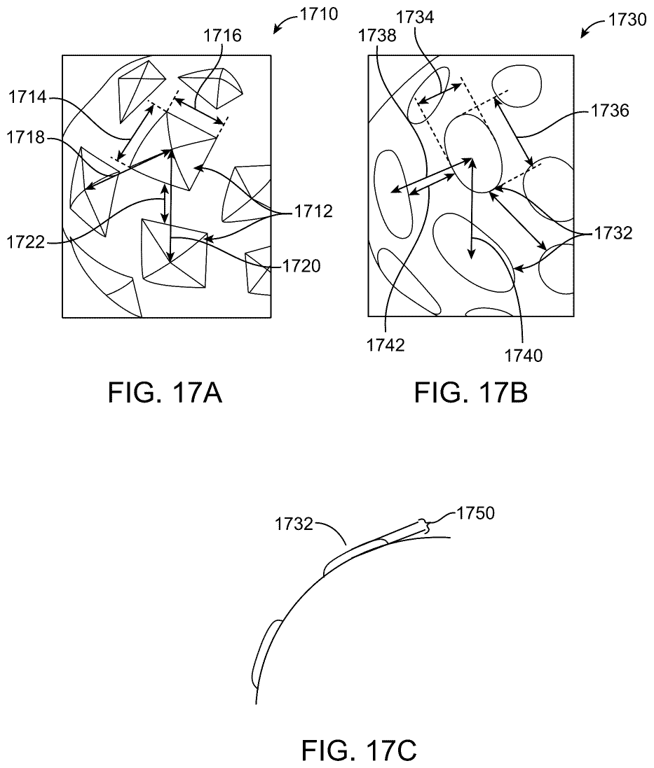

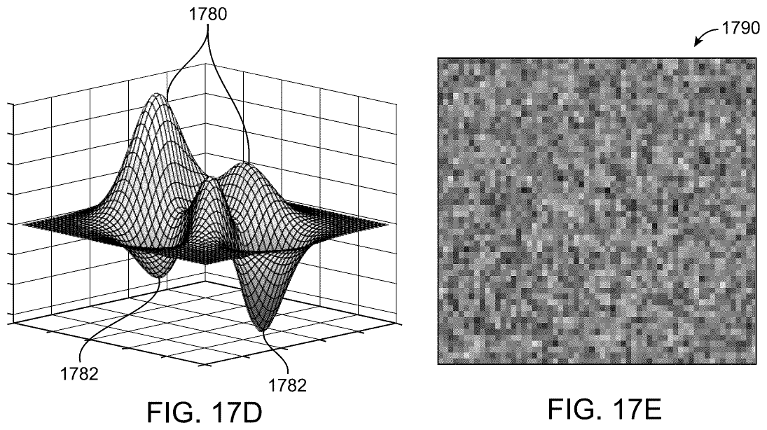

FIGS. 17A, 17B, 17C, 17D, and 17E depict examples of topographical appliance features that increase the friction between an appliance and the patient's dentition and resulting increase in retention of the appliance on the patient's dentition and improved dental treatment outcomes.

FIG. 17A depicts a knurl micro-texture 1710. In particular a checked-knurl micro-texture. The knurl micro-texture can be described as a two-dimensional grid of bumps 1712 that rise from the inner wall of a tooth receiving cavity of the dental appliance. For example, if the appliance wall is considered a flat u-v plane that hugs the tooth, the bumps 1712 sit at the intersections of the u-axis (mesio-distal) and v-axis (occluso-gingival) directions, while the z-axis points inward toward the tooth. Each individual bump 1712 occupies a footprint whose length and width may be between 0.050 mm and 1.5 mm. The bump's peak may have a height that stands 0.05 mm to 0.5 mm above the wall. This height is high enough to raise the friction coefficient but still thin enough to preserve overall shell thickness.

At the array level, bumps 1712 may repeat on a regular center-to-center pitch 1718, 1720 of 0.20 mm to 3.0 mm, preferably between 0.20 mm and 2.0 mm along both axes. This pitch equals the bump width 1714 and/or length 1716 plus a land or gap 1722 of between 0.05 mm and 2.5 mm, preferably between 0.1 mm and 1.5 mm. An area of between 0.5 mm and 2.0 mm away from gingival trim lines, incisal edges, and/or other margins may be kept clear of bumps 1712 to avoid patient discomfort and maintain aligner fit. Pitch or center to center spacing in turn governs the planar density of the features. In some embodiments, a pitch of between 1 mm and 3.0 mm may be preferred. In some embodiments, the density of the bumps may cover between 5% and 20%, preferably between 10% and 20% of the surface area of the area of the appliance to which the bumps are applied. In some embodiments, the distance between bumps may be determined based on a distance 1722 between outer perimeter of each bump, which may be in a range of between 0.1 mm and 2.5 mm, preferably in a range between 0.5 mm and 2.0 mm.

This checkered grid of topographic features 1710 depicted in FIG. 17A can be modified. For example, by rotating the grid by 45 degrees with respect to the u and v axes converts the pattern to a diamond orientation. Other rotations may also be used, such as between 5 degrees and 85 degrees, preferably between 30 degrees and 60 degrees. Offsetting alternating rows by half a pitch creates a brick-or staggered-layout that breaks up straight troughs and may distribute pressure more uniformly. Replacing the single height with two height levels wherein the height of bumps alternates between a first height and a second heigh along one or both axis, yields a dual-height texture in which taller bumps engage a tooth first while shorter bumps may aid in retention when an aligner has started to relax.

Although the topographic features in FIG. 17A are depicted as an array, such as a grid, checkered grid, etc., the topographic features may be arranged in other patterns, such as randomly. In some embodiments, the features may be applied randomly with a desired density or within a desired density range, as discussed herein.

FIGS. 17B and 17C depicts and a checkered-oval micro-texture 1730. The checked-oval micro-texture can be described as a two-dimensional grid of bumps 1732 that rise from the inner wall of a tooth receiving cavity of the dental appliance. For example, if the appliance wall is considered a flat u-v plane that hugs the tooth, the bumps 1732 sit at the intersections of the u-axis (mesio-distal) and v-axis (occluso-gingival) directions, while the z-axis points inward toward the tooth. Each individual bump 1732 occupies a footprint whose major axis 1736 and minor axis 1734 may be between 0.10 mm and 1.5 mm. The bump's peak may have a height 1750 that stands 0.01 mm to 0.2 mm above the wall. This height is high enough to raise the friction coefficient but still thin enough to preserve overall shell thickness. Where the bump merges into the wall, a tiny base fillet of roughly 25-50 μm may be added to spread stresses generated by cyclic seating of the appliance on the tooth.

At the array level, bumps 1732 may repeat on a regular center-to-center pitch 1738, 1740 of 0.20 mm to 3.0 mm, preferably between 0.20 mm and 2.0 mm along both axes. This pitch equals the bump major axis 1736 and/or minor axis 1734 plus a land or gap 1742 of between 0.05 mm and 2.5 mm, preferably between 0.1 mm and 1.5 mm. An area of at least 0.5 mm, such as between 0.5 mm and 2.0 mm, preferably 1.0 mm away from gingival trim lines, incisal edges, and/or other margins may be kept clear of bumps 1732 to avoid patient discomfort and maintain aligner fit. Pitch or center to center spacing in turn governs the planar density of the features. In some embodiments, a pitch of between 1 mm and 3.0 mm may be preferred. In some embodiments, the density of the bumps may cover between 5% and 20%, preferably between 10% and 20% of the surface area of the area of the appliance to which the bumps are applied. In some embodiments, the distance between bumps may be determined based on a distance 1742 between outer perimeter of each bump, which may be in a range of between 0.1 mm and 2.5 mm, preferably in a range between 0.5 mm and 2.0 mm.

This checkered grid 1730 depicted in FIG. 17B can be modified. For example, by rotating the grid by 45 degrees with respect to the u and v axes converts the pattern to a diamond orientation. Other rotations may also be used, such as between 5 degrees and 85 degrees, preferably between 30 degrees and 60 degrees. Offsetting alternating rows by half a pitch creates a brick-or staggered-layout that breaks up straight troughs and may distribute pressure more uniformly. Replacing the single height with two height levels wherein the height of bumps alternates between a first height and a second heigh along one or both axis, yields a dual-height texture in which taller bumps engage a tooth first while shorter bumps may aid in retention when an aligner has started to relax.

Although the topographic features in FIG. 17B are depicted as an array, such as a grid, checkered grid, etc., the topographic features may be arranged in other patterns, such as randomly. In some embodiments, the features may be applied randomly with a desired density or within a desired density range.

While various feature shapes and patterns are described herein, other feature shapes and patterns may be used. The shapes and patterns may be applied to aligner using a greyscale or other image, wherein the color and greyscale value (such as between 0 and 255 for an 8-bit image) is scaled to a height of, for example, between 0 and a maximum feature height, as discussed herein. Pixel size (length and width) may also be scaled to appropriate dimensions for a dental appliance, such as twice the minimum feature size. The height information from the image may then be applied to the geometry of the inner surface of an appliance to modify the geometry.

FIGS. 17D and 17E depicts the use of mathematic functions to alter the surface of an appliance. The mathematic function may be applied directly to the geometry or may first be used to generate a thickness map or image map, which may then be applied to the geometry, as described herein.

In some embodiments, the aligner's inner wall may be modified by a height-field defined by an equation, such as wherein z=f(u,v), which can be used to generate peaks and valleys that increase friction. For example, in some embodiments, the peaks 1780 and valleys 1782 may be generated by adding two sine waves at right angles, producing a smooth “egg-crate” array of hills and saddles whose height (or amplitude) is set by a single number and whose peak-to-peak spacing equals the chosen wavelength. Shorter wavelengths space features closer together while longer wavelengths space them further apart. In some embodiments, a radial sine or Bessel function may be used to generate rings of peaks and valleys.

In some embodiments, the surface may be seeded with random numbers or noise 1790 to which a low-pass filter may be applied to generate an isotropic roughness whose root-mean-square height controls height while a low-pass filter's cutoff frequency determines whether that peaks are sharp or smooth. In some embodiments, multiple random number functions with different low-pass or band-pass filters may be used to layer coarse ridges over fine asperities.

In some embodiments, the surface may be seeded with random numbers or noise to which a threshold may be applied. The threshold may be used to determine in which locations a feature is placed. For example, features may be placed at locations exceeding the threshold and not in locations that do not exceed the threshold. Minimum distances between features may be enforced to prevent features from being placed too close together or on top of each other. For example, when a feature is indicated to be placed less than a threshold from another features, that feature may not be placed, or may be moved to a location greater than the minimum distance. In some embodiments, features may not be placed at locations that exceed the threshold and in locations that do not exceed the threshold.

All of these mathematical surfaces may be converted to texture by sampling the function into a grayscale bitmap wherein white or a value of 255 is for high spots, black or a value of 0 is for depressions, which can then be applied to the aligner via 3D texture mapping. The bitmap's brightest value scaled to real-world dimensions determines the physical amplitude, which in a dental appliance may be between should stay between about 10 μm and 200 μm so the shell remains thin and comfortable. Horizontal scaling of the bitmap sets the wavelength or feature spacing, which may be between 0.10-0.30 mm.

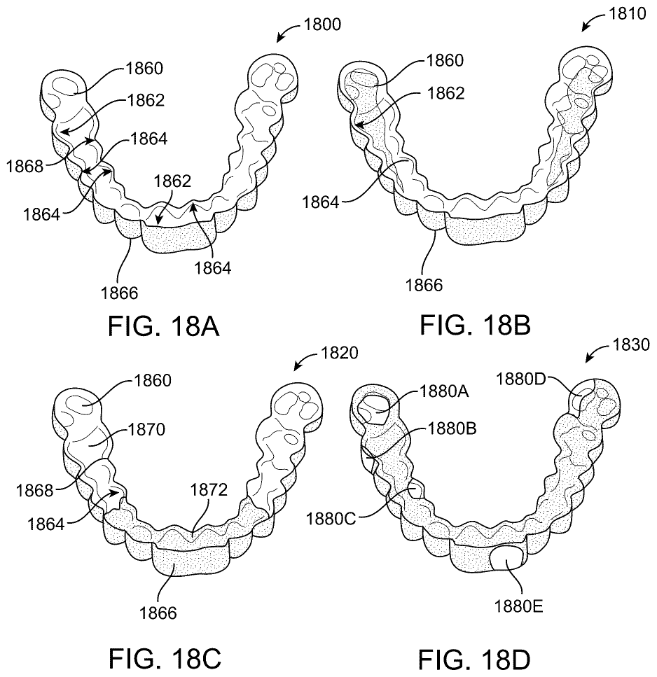

FIGS. 18A, 18B, 18C, and 18D depict topographical appliance feature placement on dental appliances, in accordance with some embodiments.

FIG. 18A depicts a full-arch dental appliance 1800 rendered in an occlusal-down perspective so that both the exterior shell 1866 and the interior, tooth-receiving cavities are visible simultaneously. The interior surfaces of the appliance carry micro-topographies.

In some embodiments, the inner buccal surfaces 1864 of both the posterior and anterior tooth receiving cavities include a micro-texture that extends from near the gingival margin up to the occlusal or incisal surface. In some embodiments, the inner buccal surfaces 1862 of both the posterior and anterior tooth receiving cavities include a micro-texture that extends from near the gingival margin up to the occlusal or incisal surface. In some embodiments, the inner occlusal and/or incisal surfaces 1860 of both the posterior and anterior tooth receiving cavities include a micro-texture that extends from the buccal side to the lingual side of the occlusal and/or incisal surface.

In some embodiments, the inner interproximal surfaces 1868 of buccal and/or lingual surfaces of the posterior and/or anterior tooth receiving cavities include a micro-texture that extends from near the gingival margin up to the occlusal or incisal surface.

FIG. 18B depicts a full-arch dental appliance 1810 rendered in an occlusal-down perspective. Similar to FIG. 18A both the exterior shell and the interior, tooth-receiving cavities are visible simultaneously and the interior surfaces of the appliance carry micro-topographies.

In some embodiments, the inner buccal surfaces 1864 of both the posterior and anterior tooth receiving cavities include a micro-texture that extends from near the gingival margin up to, but not covering, the occlusal or incisal surface. In some embodiments, the inner buccal surfaces 1862 of both the posterior and anterior tooth receiving cavities include a micro-texture that extends from near the gingival margin up to, but not covering, the occlusal or incisal surfaces 1860.

In some embodiments, the inner interproximal surfaces 1868 of buccal and/or lingual surfaces of the posterior and/or anterior tooth receiving cavities include a micro-texture that extends from near the gingival margin up to the occlusal or incisal surface.

FIG. 18C depicts a full-arch dental appliance 1820 rendered in an occlusal-down perspective Similar to FIG. 18A, both the exterior shell and the interior, tooth-receiving cavities are visible simultaneously and the interior surfaces of the appliance carry micro-topographies.

In some embodiments, the inner buccal surfaces 1864 of the posterior tooth receiving cavities 1870 include a micro-texture that extends from near the gingival margin up to the occlusal or incisal surface. In some embodiments, the inner buccal surfaces 1862 of the posterior tooth receiving cavities include a micro-texture that extends from near the gingival margin up to the occlusal or incisal surface. In some embodiments, the inner occlusal and/or incisal surfaces 1860 of the posterior tooth receiving cavities include a micro-texture that extends from the buccal side to the lingual side of the occlusal and/or incisal surface. In some embodiments, the inner interproximal surfaces 1868 of buccal and/or lingual surfaces of the posterior tooth receiving cavities include a micro-texture that extends from near the gingival margin up to the occlusal or incisal surface.

Although the appliance 1820 in FIG. 18C is depicted with posterior teeth receiving cavities having features while the anterior teeth receiving cavities do not, in some embodiments the placement of the features may be swapped. For example, in some embodiments, the inner buccal surfaces 1864 of the anterior tooth receiving cavities 1870 include a micro-texture that extends from near the gingival margin up to the occlusal or incisal surface and the inner buccal surfaces 1862 of the anterior tooth receiving cavities include a micro-texture that extends from near the gingival margin up to the occlusal or incisal surface. In some embodiments, the inner occlusal and/or incisal surfaces 1860 of the posterior tooth receiving cavities include a micro-texture.

In some embodiments, the inner interproximal surfaces 1868 of buccal and/or lingual surfaces of the posterior tooth receiving cavities include a micro-texture that extends from near the gingival margin up to the occlusal or incisal surface.

FIG. 18D depicts a full-arch dental appliance 1830 rendered in an occlusal-down perspective Similar to FIG. 18A, both the exterior shell and the interior, tooth-receiving cavities are visible simultaneously and the interior surfaces of the appliance carry micro-topographies.

The appliance 1830 includes localized areas 1880 of the interior surfaces with micro-topographic features. For example, area 1880A includes features on an occlusal surface of a posterior tooth, such as a molar. However, in some embodiments, area 1880A may be located on a pre-molar, a bicuspid, or an incisor, or other anterior or posterior occlusal or incisal location. Areas 1880B and 1880E includes features on a buccal surface of a posterior tooth, such as a molar, and an anterior tooth, such as an incisor. However, in some embodiments, area 1880B and 1880E may be located on a pre-molar, a bicuspid, or an incisor, or other anterior or posterior buccal location. Areas 1880C and 1880D include features on lingual surfaces of a molar and a premolar, respectively. However, in some embodiments, areas 1880C and 188D may be located on a pre-molar, a bicuspid, or an incisor, or other anterior or posterior lingual location.

Areas 1880 may be combined or adjacent to each other such that an area 1880 may include the buccal, lingual, and/or occlusal/incisal surface of a single tooth. In some embodiments, a single tooth may have features on the buccal and lingual sides, but not the occlusal/incisal area.

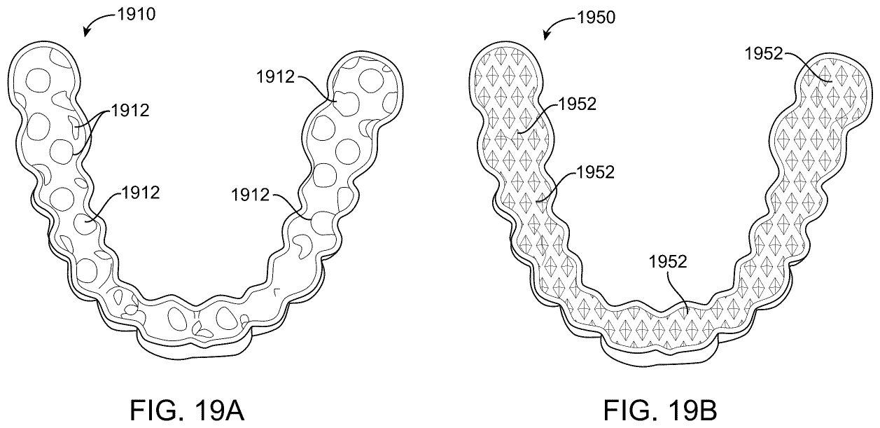

FIG. 19A depicts an example dental appliance 1910 with topographical appliance features 1912. In this aligner, the topographical appliance features 1912 are an embodiment of the checkered-oval micro-texture 1730 pattern of FIG. 17B. The pattern has been applied as an 8 mm by 8 mm texture map that is tiled on the appliance surface and includes an oval micro-texture with the same major and minor diameters, e.g., circles, of approximately 3 mm diameter.

FIG. 19B depicts an example dental appliance 1950 with topographical appliance features 1952. In this appliance, the topographical appliance features 1952 are an embodiment of the checkered knurl micro-texture 1710 pattern of FIG. 17A. The pattern has been applied as a 4 mm by 4 mm texture map that is tiled on the appliance surface and includes a diamond micro-texture with a width of approximately 2 mm and a length of approximately 4 mm.

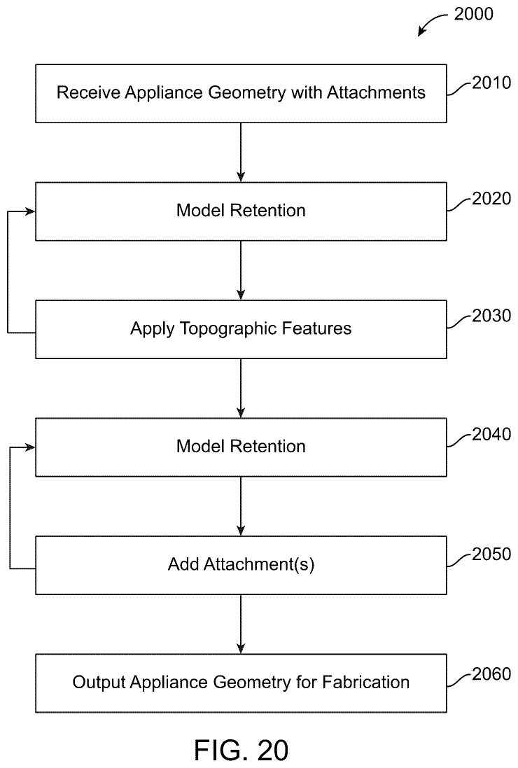

FIG. 20 depicts a method of appliance design using topographical appliance features, in accordance with some embodiments. FIG. 20 depicts a method 2000 for determining appliance geometries with one or both of topographic features and attachments to improve retention. The method 2000 may be an automated, closed-loop design routine that tries multiple surface-texture feature permutations before conceding to visible attachments, to attempt to generate a fully attachment-free appliance and before attempting to generate an appliance geometry that includes attachments and before generating an appliance geometry with visible attachments. The process may begin at step 2010, where the process receives or otherwise generates an appliance geometry for a stage-specific aligner shell along with treatment-plan data relevant to that stage, such as planned tooth positions, target retention forces, material properties, printer-resolution limits, and any clinician-defined heuristics such as avoiding anterior attachments, if possible, or other location information for the presence and/or absence of attachments and topographic features. These inputs may be stored in a design record.

In step 2020 the baseline retention of the appliance geometry is modeled, as described herein, to generate a global retention score and/or a per-tooth breakdown. If the untextured shell already meets the target retention without attachments, the algorithm outputs the geometry. Otherwise it may advance to step 2030, where the process explores micro-topographies. Here the inner surfaces of the appliance have topographic features applied to them. The topographic features may be parameterized. Parameters may include kind (for example, a checkered knurl, dimple bumps, cone field, bi-sinusoidal “egg-crate” grid, Gaussian roughness, or other type of surface feature as disused herein), size (e.g., feature footprint or wavelength, height, edge radius, draft angle, major and/or minor axis, length and/or width, and other geometric parameters), array geometry (pitch in the u- and v-directions, center-to-center spacing, orientation, and percentage coverage area, and other parameters described herein), and dentition location (which buccal, lingual, occlusal, incisal, or interproximal walls receive texture). In some embodiments, parameters may also include manufacturing parameters, such as minimum printer feature length, width, and height, minimum voxel size, etc. In some embodiments, the topographic feature parameter search space may be limited by the manufacturing parameters, such as limiting the size parameters to a multiple of the minimum manufacturing parameter size, such as no smaller than twice the minimum manufacturing parameter size

Each newly textured shell loops back to step 2020 for another retention simulation. If a particular combination of kind, size, array, and location raises the retention above the target threshold, the parameters may be stored and the method proceeds to output a geometric model of an appliance having topographic features with the particular combination of parameters. If, after exhausting its permitted iterations, no topographic solution succeeds, the algorithm may flag the case as “topography-only infeasible.” At step 2040, composite attachments enter the search space. Attachments may be introduced only on teeth whose per-tooth scores still fall short of the target retention or on teeth immediately adjacent teeth where the per-tooth score falls short. A cost function may penalize attachments on visible anterior surfaces so the process may attempt posterior or lingual surfaces first. The process may supplement earlier textures, replace them locally, or in a fully hybrid mode treat textures and attachments as simultaneous variables, wherein the method swaps, enlarges, or removes either class of feature from a location or locations as attempts to reach the retention target.

At step 2050 the retention is modeled, for example, as discussed elsewhere herein, such as with respect to step 2020. Steps 2040 and 2050 may iterate until the global and per-tooth retention targets are met and/or an objective, such as the minimization of visible attachments, stops improving, or until a user-defined compute budget, such as a number of iterations, is reached, in which case the stage is flagged for clinical review.

At step 2060, the system generates and output an appliance geometry that may include a merging of the accepted micro-textures with any attachment receiving wells. The geometry may be an STL file.

An appliance having the topographic surface features described herein may be fabricated in many ways. For example, they may be fabricated using direct fabrication, textured-mold thermoforming, post-appliance fabrication bonded textured films, and interior subtractive fabrication.

In some embodiments of direct fabrication, the appliance shell may first be modeled without topographic features and then, based on the treatment planning, as described herein, the topographic features may be added or otherwise is merged directly into the mesh of the 3D model, such as by using a grayscale bitmap, through an analytic or mathematic height-field application to the surface of the appliance model or otherwise. In some embodiments, the application geometry may be generated with the features, without first being generated without the topographic features. The model of the appliance with the topographic features may be output and fabricated, using direct fabrication, based on the model.

In some indirect fabrication methods, such as thermoforming, the appliance shell may first be modeled without topographic features and then, based on the treatment planning, as described herein, the topographic features may be added or otherwise is merged directly into the mesh of the 3D model, such as by using a grayscale bitmap, through an analytic or mathematic height-field application to the surface of the appliance model or otherwise. In some embodiments, the application geometry may be generated with the features, without first being generated without the topographic features. A mold may be generated based on the 3D model of the appliance and a sheet of material may be thermoformed over the mold and then trimmed to form the appliance.

In some embodiments, a sheet of material having topographic features may be adhered or bonded to a physical appliance. In such an embodiment, a model with or without topographic features may be generated, as described herein, but the appliance is initially fabricated without or with less than all of the topographic features for the appliance. For example, features that printers or molds cannot resolve, such as those with sizes less than a minimum feature size of a molding process or direct fabrication process, such features can be added as thin, pre-textured stickers. For example, a thin substrate, such as medical-grade PET or PU film may be roller-embossed or otherwise have topographic features discussed herein formed on a first surface or side of the substrate. The other side of the substrate may receive pressure sensitive adhesive, such as a pressure-sensitive acrylic or silicone contact adhesive that bonds to the aligner's cured resin or thermoform body. The substrate may be cut, such as die-cut into and adhered or bonded onto the inner wall of the appliance. In some embodiments, light cured adhesives may be used to bond the substrate onto the aligner in locations indicated by the aligner design or treatment planning process or as desired by a dental professional. Aligners and associated topographic adhesive materials may be fabricated and sent to a dental professional who then applies the topographic features to the appliance.

In some emblements, subtractive fabrication may be used to form topographic features on an appliance. For example, an appliance may be fabricated with locations for topographic features having a greater thickness than locations without topographic features, such as between 0.2 and 0.8 mm thicker. Then, material may be removed from the thicker areas to form the topographic features, such as through etching, including chemical etching wherein a mask is used to along with chemical etching to form the topographic features. In some embodiments, ablation, such as laser ablation may be used to remove material and form the topographic features. In some embodiments, machining may remove material from the appliance to form the topographic features.

In some embodiments, the method 2000 may be used within the method 100. For example, method 100, which generates, evaluates, and finalizes an appliance for each stage of an orthodontic treatment plan, step 160 is where the aligner geometry is modified to adjust retention for the planned arch. At that step or elsewhere, the method 100 may include the method 2000, for example, as sub-routine. In some embodiments, the treatment plan information for a stage of treatment, including an initial appliance geometer and other information discussed herein, such as, the three-dimensional tooth arrangement, the force-system targets for each tooth, material options, pixel size of the chosen printer, patient comfort limits, and any doctor-specified preferences, may be used by method 2000.

Method 2000 then runs its iterative loop as described herein, which may include first simulating baseline retention, then exploring a design space of surface-topography permutations including one or both of texture-mapped patterns such as checkered knurls and mathematically generated landscapes such as bi-sinusoidal grids. Each candidate is converted into an appliance geometry and tested for retention. If a purely topographic solution meets the desired target, such as a retention or force application target, the appliance geometry is generated and the process returns to method 100. If no combination of kind, size, array geometry, and dentition-location achieves the goal, method 2000 continues to its attachment phase, adding and/or removing or moving attachments, with posterior-and-lingual placements scored more favorably than labial-anterior ones. Once the global and per-tooth retention targets are satisfied, either via textures alone or via a hybrid of textures and attachments, the geometry is passed back to method 100.

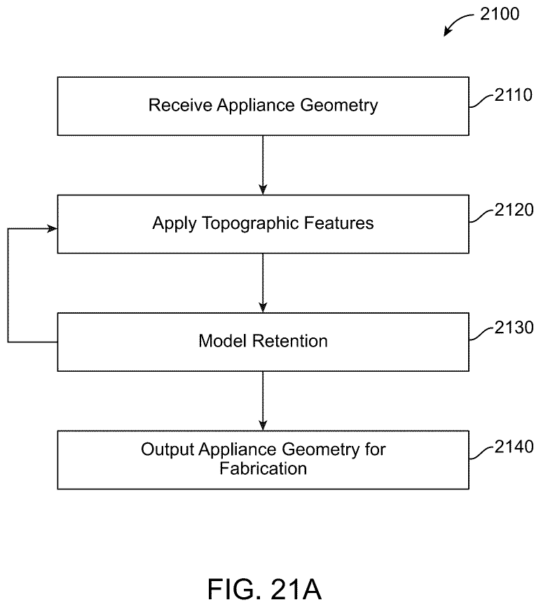

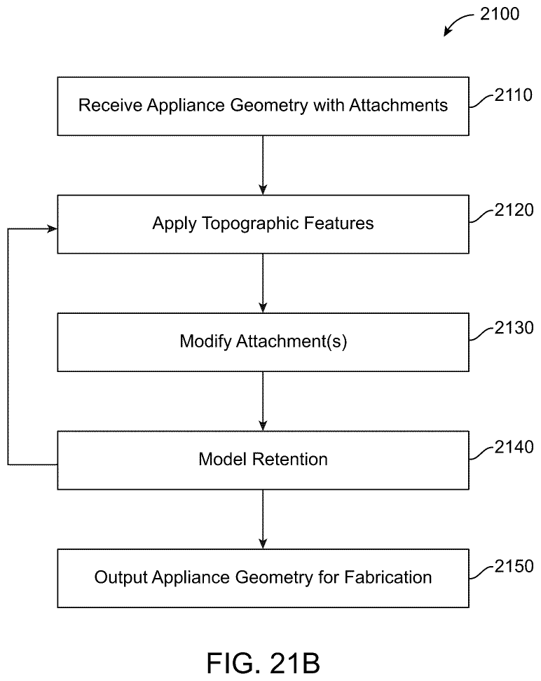

FIG. 21A depicts a method 2100 for designing a dental appliance with topographic features. The method may begin at step 2110, by receiving or otherwise generating a treatment plan, including at least one stage-specific appliance shell together with additional parameters that may influence retention, such as three-dimensional tooth positions, retention and/or orthodontic tooth movement forces, material and printer properties and/or limits, and any clinician preferences such as, maintaining anterior teeth attachment free. At step 2120 the method may apply candidate micro-topographies on selected inner surfaces of the appliance. These micro-topography textures may be bitmap patterns (checkered knurls, dimple bumps, cone lattices) or analytically generated height-fields (bi-sinusoidal “egg-crate” grids Gaussian roughness, etc), or 3D models of textures. Each texture may be parameterized by kind, peak height, footprint or wavelength, pitch in the u- and v-directions, coverage mask, and dentition-location mask, as discussed herein, and may be applied to the mesh geometry via, for example, a 3-D-Texture engine.

At step 2130 the modified appliance is modeled for retention and a global retention score and per-tooth retention metric may be generated, as described herein, such as with respect to FIG. 1. If the retention target or orthodontic force targets is not met, the method may modify the topographic features, iterating through steps 2120 and 2130 in a loop that iteratively refines the micro-texture parameters.

As the loop progresses, topographies may be adjusted, such as by raising amplitude, tightening pitch, enlarging coverage, redirecting orientation, or by modifying any of the other parameters discussed herein, so that the frictional contribution of the surface microtopography can compensate for the retention and/or orthodontic forces once provided by the attachment. The process may end when the retention and/or orthodontic force target or targets (such as for an individual tooth, each individual tooth, or the aligner/arch is satisfied or when an iteration limit is reached.

At step 2140, the method may output an appliance model for fabrication. The applicant model may merge the micro-topographies with the residual attachment receiving cavities, if any.

FIG. 21B depicts a method 2102 for attachment-reduction for a dental appliance. The method may begin at step 2112, by receiving or otherwise generating a treatment plan stage-specific aligner shell that already includes one or more attachments prescribed in the treatment plan, together with additional parameters that may influence retention, such as three-dimensional tooth positions, retention and/or orthodontic tooth movement forces, material and printer properties and/or limits, and any clinician preferences such as, maintaining anterior teeth attachment free. In step 2122 the method may apply candidate micro-topographies on selected inner surfaces of the appliance. These micro-topography textures may be bitmap patterns (checkered knurls, dimple bumps, cone lattices) or analytically generated height-fields (bi-sinusoidal “egg-crate” grids Gaussian roughness, etc), or 3D models of textures. Each texture may be parameterized by kind, peak height, footprint or wavelength, pitch in the u- and v-directions, coverage mask, dentition-location mask, etc., as discussed herein, and may be applied to the mesh geometry via, for example, a 3D-Texture engine.

At step 2132 the method may then select one attachment at a time for removal or replacement. For example, starting with anterior and/or buccal attachments that are visible during a natural smile of the patient. The method may remove the attachment, replace it with a smaller, less visible attachment, or move the attachment to an adjacent tooth and then update the shell geometry.

At step 2142 the modified appliance is modeled for retention and a global retention score and per-tooth retention metric may be generated, as described herein, such as with respect to FIG. 1. If retention and orthodontic force application targets are still met, the attachment may be deemed unnecessary and remains deleted. If the retention target or orthodontic force targets is not met, the method restores that attachment and/or modifies the topographic features, and/or moves on to the next candidate attachment for removal, iterating through steps 2122, 2132, 2142 in a loop that iteratively refines both the micro-texture parameters and the attachment set.

As the loop progresses, topographies may be adjusted, such as by raising amplitude, tightening pitch, enlarging coverage, redirecting orientation or by modifying any of the other parameters discussed herein, each time an attachment is removed, so that the frictional contribution of the surface microtopography can compensate for the retention and/or orthodontic forces once provided by the attachment. The process may end when either every attachment has been removed while retention and/or orthodontic force targets remain satisfied, yielding a fully attachment-free solution, when no further attachment can be eliminated without dropping below the target retention and orthodontic forces, or when an iteration limit is reached, in which case the smallest subset of attachments or a smallest subject set of buccally located anterior attachments is retained.

At step 2152, the method may output an appliance model for fabrication. The applicant model may merge the micro-topographies with the residual attachment receiving cavities, if any.

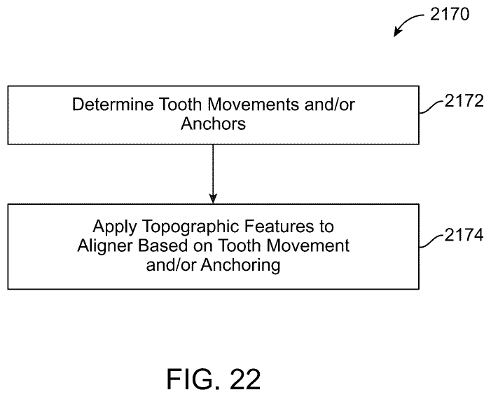

FIG. 22 depicts a method 2170 for use in generating dental appliances and in orthodontic and other dental treatments and treatment planning processes. The method 2170 aids in determining where to place topographic features on an appliance the parameters of the topographic features, such as size, shape, density, arrangements, etc., to increase surface friction between the patient's dentition and the appliance. In a treatment planning process, such as those described herein, a sequence of tooth movement and/or jaw movement stages are generated to move a patient's teeth from an initial arrangement towards a final arrangement. The treatment planning process may include moving some teeth (and determining a force system to apply to a tooth to move it) and using some teeth as anchoring teeth (and determining a force system to apply to an anchor tooth) for one or more, such as each stage of a dental treatment.

As part of a treatment planning and/or appliance design process, such as described herein, the process may include a step 2172 that determine which teeth are moving during a stage of treatment, such as those receiving forces to intended cause translation, rotation, or inclination changes to the teeth and which teeth are anchoring teeth that may act to as an anchor, such as by accepting reciprocal forces, but with little to no movements. In some embodiments, the method may include associating the movement of a tooth (or group of teeth) and the teeth that act as an anchor of the movement of the tooth (or group of teeth).

At step 2174, the process may apply topographical features, such as those applied herein, for example, according to the methods described herein, onto the interior surfaces of the appliance to increase local static and kinetic friction or for other uses in one or more discrete surface zones or areas that correspond to the anchoring and/or moving teeth identified at step 2172. Feature parameters, such as peak to valley amplitude, root mean square roughness, pattern geometry, and other feature parameters discussed herein may be determined. The determination may be based on target retention forces exceed the predicted reaction forces on anchoring teeth with a safety factor while keeping appliance application and removal forces within patient tolerable limits.

At step 2174, one or more movements or other treatments may be determined or others identified and a corresponding change in or appliance of surface topography may be applied or provided as feedback for application in the treatment planning process. In some embodiments, a vertical movement, such as intrusion or extrusion, may be identified for a tooth and a topographic pattern may be applied to the teeth adjacent the vertically moving teeth. For example, when the central and lateral incisors are planned for intrusion or extrusion, the method may apply or suggest the application of a surface topology pattern, as discussed herein, to the buccal and lingual sidewalls of the neighboring canines to create a stable anchor for the vertical movement without visible attachments.

In some embodiments, transverse movements of one or more teeth may be identified and the method may apply surface topographic modifications to the teeth adjacent the transversally moving teeth. For example, if a first premolar is being translated buccally, surface topology features, such as those described herein, may be applied to the buccal, lingual, and/or interproximal areas of the inner surface of the appliance on the teeth receiving cavities of the first and second molars to anchor the transverse movement.

In some embodiments, one or more teeth may be determined or otherwise identified as having sagittal movements, such as an uprighting. When uprighting is identified in a tooth, the teeth receiving cavities of the application adjacent to the tooth undergoing uprighting movement may have topographical features added thereon, as described herein. For example, during distal uprighting of a tipped second molar, the method may apply topographic surface features to the two adjacent premolars to anchor the sagittal moment.

In some embodiments, one or more rounded teeth may be determined or otherwise identified as having a rotational movement. Because frictional contact is the primary driver of rotational torque on rounded teeth when orthodontic tooth movement is accomplished without attachments, such as canines and premolars, the method may apply topographic features directly on those teeth's buccal, lingual, and occlusal walls to increase friction between the appliance and the tooth. In some embodiments, rotational movement of rounded teeth, such as canines and premolars, may be especially challenging. In some embodiments, movement of rounded or other shaped teeth may be accomplished using a combination of attachments (with corresponding attachment receiving cavities on the appliance) in combination with topographic features on the interior surface of canine, premolar, or other tooth receiving cavities.

In some embodiments, the retention of the entire appliance is to be increased. For example, some patients may have poor aligner retention, the treatment planning process may identify poor retention, or a dentist may prefer greater retention, for example. At step 2174, the method and apply to suggest application of topographic features as discussed herein on the inner surface of the appliance, such as the entire inner surface, on lingual and buccal sides of inner surface, on inner surface of molars and bicuspids, and/or select surfaces on various teeth, such as anchoring teeth.

In some embodiments, during treatment planning, and/or retention or other appliance retention and/or simulation modeling process, it may be determined that predicts that the appliance may push itself off, in what may be called the watermelon seed effect. In such embodiments, surface topographic features, such as those discussed herein, may be applied to the anterior teeth and teeth anchoring the forces applies to the anterior teeth, as discussed herein.

In some embodiments, during the treatment planning and/or appliance design process, a discolored or missing anterior tooth may be identified. Topographic features on an appliance may alter the opacity of the appliance. Dense and small topographic features may be added to an appliance at the location of missing or erupting tooth to decrease the opacity of the appliance at that location and mask the gap in the detention at the location of the missing or erupting tooth. Such topographic features may have length and width dimensions of less than 1 mm, preferably less than 0.5 mm. In some embodiments, the length and width may be 1, 2, 3, 4, or 5 times the minimum feature size of a fabrication machine. In some embodiments, the features may be placed next to each other with little to no gap between features, such as immediately adjacent or at or below 1 times or at or below 2 times the minimum feature size of the fabrication machine.

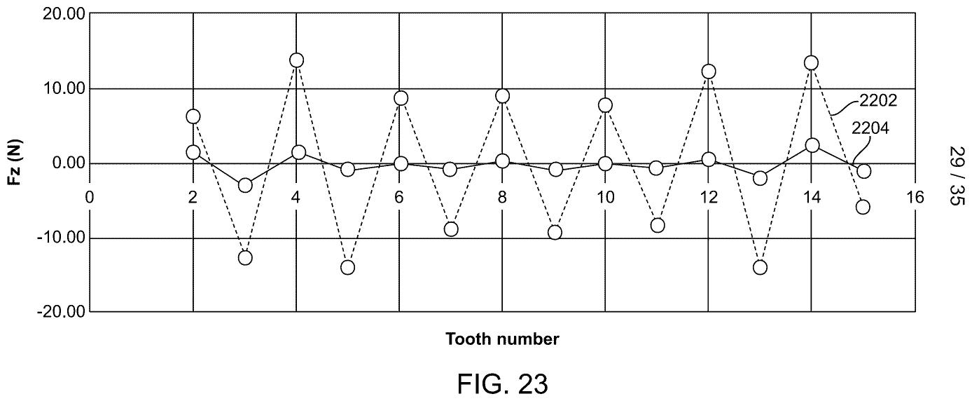

FIG. 23 depicts simulated results of retention improvement using topographical appliance features discussed herein. The graph depicts the Fz retention force (force in the occlusal-gingival direction) measured using the methods described herein of a normal appliance 2204 without the surface topographic modification discussed herein and an appliance 2202 having the same geometry as the appliance 2202, but with the topographic surface modification discussed herein. The appliance 2202, having a design based on the features and methods discussed herein, provided exceptional retention, far exceeding the retention of an unmodified appliance.

FIG. 24 Computer-Readable Medium 2410, Patient Engagement System 2420, Oral State Capture System(s) 2430, Treatment Planning System(s) 2440, Treatment Management System(s) 2450, and Appliance Fabrication System(s) 2460.

Computer-Readable Medium 2410 includes any computer-readable medium and may include a non-transitory computer readable medium comprising instructions for carrying out any of the processes and methods discussed herein, which may be carried out by one or more of the Patient Engagement System 2420, Oral State Capture System(s) 2430, Treatment Planning System(s) 2440, Treatment Management System(s) 2450, and Appliance Fabrication System(s) 2460.

Patient Engagement System 2420 can include any system to engage patients. It can be on a mobile device, laptop, or other electronic device associated with a patient, a prospect for treatment, or person evaluating treatment. It can include tools to visualize end results of treatment, clinically (by showing how a treatment plan would look on a person), non-clinically (by showing how model dentition would look on a person), or other ways. Patient Engagement System can include lead generation software, such as a mobile application configured to encourage prospects to get treatment.

Oral State Capture System(s) 2430 can include any systems to capture the state of a person's oral cavity, such as in method 100 and the other methods described herein. It can include machinery and/or software to collect intraoral scans, x-rays, sensor data from ECI systems, 2D photos taken from a phone or other camera, CBCT data, etc.

Treatment Planning Systems may provide treatment plans, treatment recommendations, orthodontic/restorative integration capabilities, and/or other capabilities, such as discussed with respect to method 100 and the other methods described herein. Treatment Planning Systems may, in some implementations: take in representations of dentition, identify (through human activities and/or automation) orthodontic/restorative treatments to dentition, provide staging/intermediate positioning/final positioning capabilities of orthodontic/restorative treatments, receive and/or process modifications from the Treatment Management Systems, provide updated treatments, support appliance design, etc. In some implementations, Treatment Planning Systems are like Align's Treat software and implement the end-to-end digital treatment planning workflow that software does.

Treatment Management Systems 2450 can include software that allows clinicians to manage treatment plans. Treatment Management Systems can collect a state of an oral cavity from Oral State Capture System(s) and/or other input modalities. Treatment Management Systems can further provide a state of an oral cavity, with or without manipulations and/or annotations, to Treatment Planning Systems. In various implementations, Treatment Management Systems allow clinicians incorporate treatment preferences (e.g., stages of a treatment plan to perform IPR; whether, when and/or where orthodontic attachments; selection, increase, decrease, etc. of stages of a treatment plan; specific preferences of a clinician to treat specific conditions; etc.). Treatment Management Systems can submit a person's case (e.g., information related to a state of an oral cavity with or without treatment preferences, etc.) to Treatment Planning Systems for a treatment plan for a person's dentition. In various implementations, Treatment Management Systems allow a clinician to approve, deny, modify, etc. a proposed treatment plan.

The Appliance Fabrication Systems 2460 may include systems that allow appliance design and/or fabrication. These could control direct fabrication of dental appliances (e.g., by 3D printing appliances) and/or indirect fabrication of direct appliances (e.g., by 3D printing molds over which appliances are subsequently thermoformed). In some implementations, Appliance Fabrication Systems include software to identify geometries of appliances to be generated. Appliance Fabrication Systems can include tools to model and/or optimize retention of dental appliances given various factors.

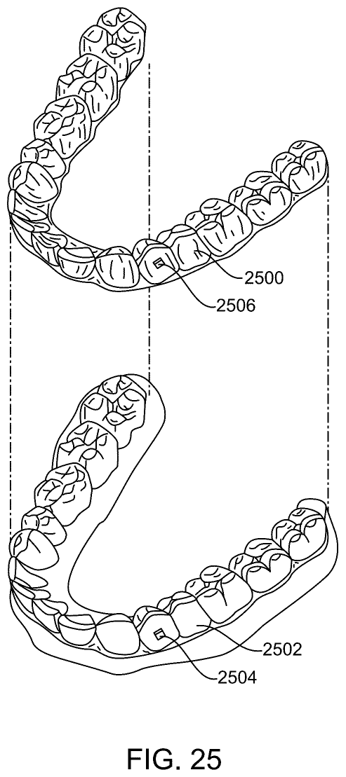

FIG. 25 illustrates an exemplary tooth repositioning appliance 2500, such as an aligner that can be worn by a patient in order to achieve an incremental repositioning of individual teeth 2502 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct formation techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 2504 on teeth 2502 with corresponding receptacles or apertures 2506 in the appliance 2500 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

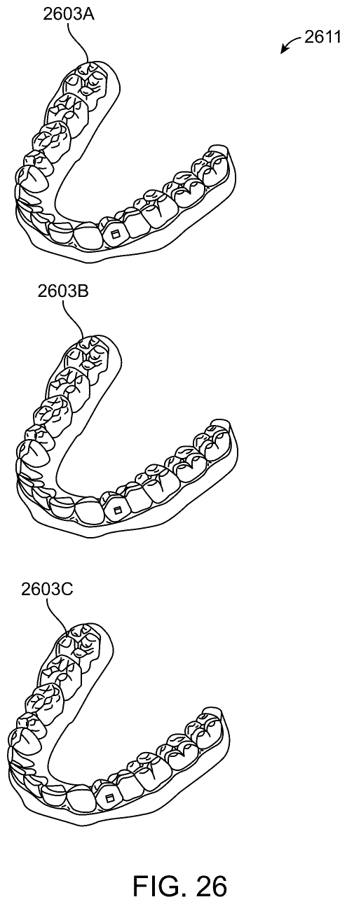

FIG. 26 illustrates a tooth repositioning system 2600 including a plurality of appliances 2603A, 2603B, 2603C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 2600 can include a first appliance 2603A corresponding to an initial tooth arrangement, one or more intermediate appliances 2603B corresponding to one or more intermediate arrangements, and a final appliance 2603C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

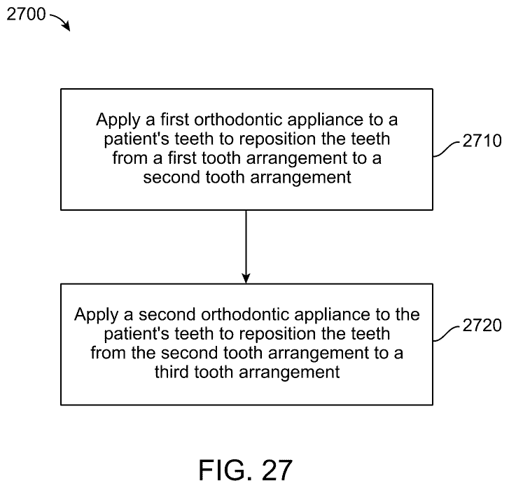

FIG. 27 illustrates a method 2700 of orthodontic treatment using a plurality of appliances, in accordance with many embodiments. The method 2700 can be practiced using any of the appliances or appliance sets described herein. In step 2710, a first orthodontic appliance is applied to a patient's teeth in order to reposition the teeth from a first tooth arrangement to a second tooth arrangement. In step 2720, a second orthodontic appliance is applied to the patient's teeth in order to reposition the teeth from the second tooth arrangement to a third tooth arrangement. The method 2700 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

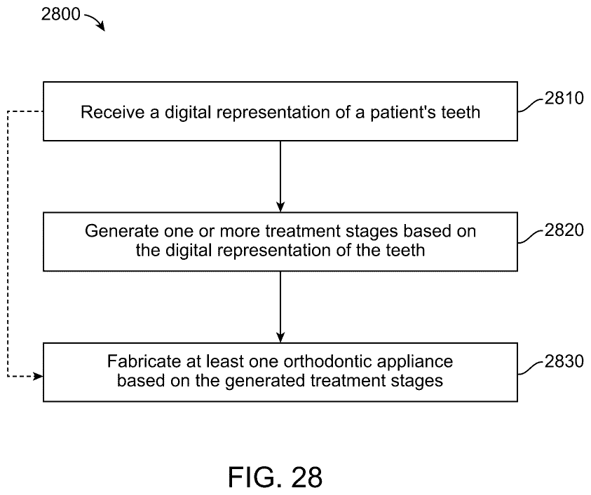

FIG. 28 illustrates a method 2800 for digitally planning an orthodontic treatment and/or design or fabrication of an appliance, in accordance with many embodiments. The method 2800 can be applied to any of the treatment procedures described herein and can be performed by any suitable data processing system. Any embodiment of the appliances described herein can be designed or fabricated using the method 2800.

In step 2810, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

The digital representation may include a digital model of the patient's upper or lower jaw generated by an oral state capture device or system.

In step 2820, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In step 2830, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

In some instances, staging of various arrangements or treatment stages may not be necessary for design and/or fabrication of an appliance. As illustrated by the dashed line in FIG. 28, design and/or fabrication of an orthodontic appliance, and perhaps a particular orthodontic treatment, may include use of a representation of the patient's teeth (e.g., receive a digital representation of the patient's teeth 2810), followed by design and/or fabrication of an orthodontic appliance based on a representation of the patient's teeth in the arrangement represented by the received representation.

FIG. 29 is a simplified block diagram of a data processing system 2900 that may be used in executing methods and processes described herein. The data processing system 2900 typically includes at least one processor 2902 that communicates with one or more peripheral devices via bus subsystem 2904. These peripheral devices typically include a storage subsystem 2906 (memory subsystem 2908 and file storage subsystem 2914), a set of user interface input and output devices 2918, and an interface to outside networks 2916. This interface is shown schematically as “Network Interface” block 2916, and is coupled to corresponding interface devices in other data processing systems via communication network interface 2924. Data processing system 2900 can include, for example, one or more computers, such as a personal computer, workstation, mainframe, laptop, and the like.

The user interface input devices 2918 are not limited to any particular device, and can typically include, for example, a keyboard, pointing device, mouse, scanner, interactive displays, touchpad, joysticks, etc. Similarly, various user interface output devices can be employed in a system of the invention, and can include, for example, one or more of a printer, display (e.g., visual, non-visual) system/subsystem, controller, projection device, audio output, and the like.

Storage subsystem 2906 maintains the basic required programming, including computer readable media having instructions (e.g., operating instructions, etc.), and data constructs. The program modules discussed herein are typically stored in storage subsystem 2906. Storage subsystem 2906 typically includes memory subsystem 2908 and file storage subsystem 2914. Memory subsystem 2908 typically includes a number of memories (e.g., RAM 2910, ROM 2912, etc.) including computer readable memory for storage of fixed instructions, instructions and data during program execution, basic input/output system, etc. File storage subsystem 2914 provides persistent (non-volatile) storage for program and data files, and can include one or more removable or fixed drives or media, hard disk, floppy disk, CD-ROM, DVD, optical drives, and the like. One or more of the storage systems, drives, etc. may be located at a remote location, such coupled via a server on a network or via the internet/World Wide Web. In this context, the term “bus subsystem” is used generically so as to include any mechanism for letting the various components and subsystems communicate with each other as intended and can include a variety of suitable components/systems that would be known or recognized as suitable for use therein. It will be recognized that various components of the system can be, but need not necessarily be at the same physical location, but could be connected via various local-area or wide-area network media, transmission systems, etc.

Scanner 2920 includes any means for obtaining a digital representation (e.g., images, surface topography data, etc.) of a patient's teeth (e.g., by scanning physical models of the teeth such as casts 2921, by scanning impressions taken of the teeth, or by directly scanning the intraoral cavity), which can be obtained either from the patient or from treating professional, such as an orthodontist, and includes means of providing the digital representation to data processing system 2900 for further processing. Scanner 2920 may be located at a location remote with respect to other components of the system and can communicate image data and/or information to data processing system 2900, for example, via a network interface 2924. Fabrication system 2922 fabricates appliances 2923 based on a treatment plan, including data set information received from data processing system 2900. Fabrication machine 2922 can, for example, be located at a remote location and receive data set information from data processing system 2900 via network interface 2924.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.
Clause 1. A method for designing dental appliances: receiving a 3D model of a patient's oral cavity; determining a target arrangement of teeth of the patient from the 3D model; generating a treatment plan for moving teeth of a patient from an initial position towards and final position over a plurality of stages; generating a 3D model of a first dental appliance; modeling retention of the first dental appliance for a first stage of the treatment plan based on the 3D model of the first dental appliance and a model of an arch of the patient for the first stage of the treatment plan to determine a first retention; modifying the 3D model of the first dental appliance to include topographic features on an interior surface of the first dental appliance to generate a modified 3D model of the first dental appliance; and outputting the modified 3D model of the first dental appliance for fabrication and physical dental appliance.
Clause 2. The method of clause 1, further comprising: determining that a tooth moves during the first stage of treatment, and wherein modifying the 3D model of the first dental appliance to include topographic features includes adding topographic features on tooth receiving cavities of the appliance immediately adjacent the tooth that moves.
Clause 3. The method of clause 1, further comprising: determining a tooth with the least retention based the modeling of retention of the first dental appliance, and wherein modifying the 3D model of the first dental appliance to include the topographic features includes modifying the tooth receiving cavities of immediately adjacent to the tooth with the least retention to include the topographic features.
Clause 4. The method of clause 1, wherein modifying the 3D model of the first dental appliance to include the topographic features includes generating an array of topographic features.
Clause 5. The method of clause 4, wherein generating the array of topographic features includes generating the array based on topographic feature parameters.
Clause 6. The method of clause 5, wherein the topographic feature parameters include one or more of height, column separation distance, and row separation distance.
Clause 7. The method of clause 5, wherein the topographic feature parameters include one or more of width, length, major axis diameter, and minor axis diameter.
Clause 8. The method of clause 5, wherein the topographic feature parameters include a density of the topographic features.
Clause 9. The method of clause 5, wherein the topographic feature parameters include fabrication machine parameters.
Clause 10. The method of clause 9, wherein the topographic feature parameters include fabrication machine parameters.
Clause 11. The method of clause 10, wherein fabrication machine parameters include a minimum feature size and the topographic features have a length and width of between 2 and 5 times the minimum feature size.
Clause 12. The method of clause 10, wherein fabrication machine parameters include a minimum feature size and the topographic features have a length and width of between 1 and 30 times the minimum feature size.
Clause 13. The method of clause 10, wherein fabrication machine parameters include a minimum feature size and the topographic features have a length and width of between 0.05 mm and 1.5 mm the minimum feature size.
Clause 14. A method for designing dental appliances: receiving a 3D model of a patient's oral cavity; determining a target arrangement of teeth of the patient from the 3D model; generating a treatment plan for moving teeth of a patient from an initial position towards and final position over a plurality of stages; generating a 3D model of a first dental appliance system; modifying the 3D model of the first dental appliance to include topographic features on an interior surface of the first dental appliance to generate a modified 3D model of the first dental appliance; and modeling retention of the first dental appliance for a first stage of the treatment plan based on the modified 3D model of the first dental appliance and a model of an arch of the patient for the first stage of the treatment plan to determine a first retention; determine, based on the modeling, whether or not the retention is sufficient for the first stage of treatment; and outputting the modified 3D model of the first dental appliance for fabrication and physical dental appliance.
Clause 15. The method of clause 14, wherein generating a 3D model of a first dental appliance system includes generating a 3D model of a first dental appliance system including one or more attachments.
Clause 16. The method of clause 15, further comprising: removing a first of the one or more attachments prior to the modifying of the 3D model and the modeling retention of the first dental appliance; determining that the retention is sufficient; removing a second of the one or more attachments; modeling, for a second time, retention of the first dental appliance for the first stage of the treatment plan based on the modified 3D model of the first dental appliance and the model of the arch of the patient for the first stage of the treatment plan to determine a first retention.
Clause 17. The method of clause 15, further comprising: removing a first of the one or more attachments prior to the modifying of the 3D model and the modeling retention of the first dental appliance; determining that the retention is insufficient; replacing the first of the one or more attachments; removing a second of the one or more attachments; modeling, for a second time, retention of the first dental appliance for the first stage of the treatment plan based on the modified 3D model of the first dental appliance and the model of the arch of the patient for the first stage of the treatment plan to determine a first retention.
Clause 18. The method of clause 16, further comprising: determining that a tooth moves during the first stage of treatment, and wherein modifying the 3D model of the first dental appliance to include topographic features includes adding topographic features on tooth receiving cavities of the appliance immediately adjacent the tooth that moves.
Clause 19. The method of clause 16, further comprising: determining a tooth with the least retention based the modeling of retention of the first dental appliance, and wherein modifying the 3D model of the first dental appliance to include the topographic features includes modifying the tooth receiving cavities of immediately adjacent to the tooth with the least retention to include the topographic features.
Clause 20. The method of clause 16, further comprising: determining a tooth with the least retention based the modeling of retention of the first dental appliance, and wherein modifying the 3D model of the first dental appliance to include the topographic features includes modifying the tooth receiving cavity of the tooth with the least retention to include the topographic features.
Clause 21. The method of clause 16, wherein modifying the 3D model of the first dental appliance to include the topographic features includes generating an pattern of topographic features.
Clause 22. The method of clause 16, wherein modifying the 3D model of the first dental appliance to include the topographic features includes generating a random pattern of topographic features.
Clause 23. The method of clause 20, wherein generating the array of topographic features includes generating the array based on topographic feature parameters.
Clause 24. The method of clause 21, wherein the topographic feature parameters include one or more of height, column separation distance, and row separation distance.
Clause 25. The method of clause 21, wherein the topographic feature parameters include one or more of width, length, major axis diameter, and minor axis diameter.
Clause 26. The method of clause 21, wherein the topographic feature parameters include a density of the topographic features.
Clause 27. The method of clause 21, wherein the topographic feature parameters include fabrication machine parameters.
Clause 28. The method of clause 25, wherein the topographic feature parameters include fabrication machine parameters.
Clause 29. The method of clause 26, wherein fabrication machine parameters include a minimum feature size and the topographic features have a length and width of between 1 and 10 times the minimum feature size.
Clause 30. The method of clause of any one of the preceding clauses, further comprising:
fabricating the first dental appliance based on the modified 3D model.
Clause 31. The method of clause 28, wherein fabricating the first dental appliance includes directly fabricating the first dental appliance.
Clause 32. The method of clause 28, wherein fabricating the first dental appliance includes: directly fabricating a mold for the first dental appliance; and thermoforming a sheet of polymeric material over the mold to form the first dental appliance.
Clause 33. The method of clause 28, wherein fabricating the first dental appliance includes: fabricating the first dental appliance without the topographic features; and modifying the first dental appliance to form the topographic features.
Clause 34. The method of clause 31, wherein modifying the first dental appliance to form the topographic features includes: subtractive fabricating the topographic features on the dental appliance.
Clause 35. The method of clause 31, wherein modifying the first dental appliance to form the topographic features includes: adhering a substrate having the topographic features onto the dental appliance.
Clause 36. A system for generating dental appliances, comprising: one or more processors and memory comprising instructions that when executed by one or more of the processors causes the system to carry out the method of any one of clauses the preceding clauses.
Clause 37. A dental appliance made by the process of any one of the preceding clauses.
Clause 38. A dental appliance comprising: a plurality of tooth receiving cavities shaped to receiving a dentition of a patient dentition, wherein a first tooth receiving cavity of the plurality of tooth receiving cavities shaped to impart an orthodontic tooth movement force on a first tooth of the patient to move the first tooth from a first position towards a second position; and a first array of topographic features on an inner surface of second and third tooth receiving cavities of the plurality of tooth receiving cavities immediately adjacent to the first tooth receiving cavity.
Clause 39. The dental appliance of clause 36, wherein the first tooth receiving cavity does not include topographic features.
Clause 40. The dental appliance of clause 36, wherein the topographic features are individual bumps.
Clause 41. The dental appliance of clause 38, wherein the second and third tooth receiving cavities have a bump density of at least 5 bumps per square centimeter.
Clause 42. The dental appliance of clause 39, wherein the bumps have a length, width, or diameter of between 2 mm and 4 mm.
Clause 43. The dental appliance of clause 40, wherein the bumps extend from the inner surface of the appliance with a height of less than 0.2 mm.
Clause 44. The dental appliance of clause 38, wherein the second and third tooth receiving cavities have a bump density of at least 50 bumps per square centimeter.
Clause 45. The dental appliance of clause 42, wherein the bumps have a length, width, or diameter of between 0.1 mm and 1 mm.
Clause 46. The dental appliance of clause 43, wherein the bumps extend from the inner surface of the appliance with a height of less than 0.2 mm.
Clause 47. The dental appliance of clause 38, wherein the array has columns and rows of individual bumps, wherein a center-to-center distance between columns is between 1 and 5 mm and a center-to-center distance between rows of individual bumps is between 1 and 5 mm.
Clause 48. A method for designing dental appliances: receiving a 3D model of a patient's oral cavity; determining a target arrangement of teeth of the patient from the 3D model; generating a treatment plan for moving teeth of a patient from an initial position towards and final position over a plurality of stages; modeling a retention of a first dental appliance for a first stage of the treatment plan on the model of an arch of the patient for the first stage of the treatment plan to determine a first retention of the first dental appliance on the model of the arch of the patient; modeling the retention of a second dental appliance on a retentive model of an arch of the patient to determine a second retention of the second dental appliance on the retentive model of the arch of the patient, the retentive model of the arch including attachments added to the teeth of the patient; generating a retention difference between the first retention of the first dental appliance on the model of the arch of the patient and the second retention of the second dental appliance on the retentive model of the arch of the patient; and modifying the first dental appliance to increase retention of the first dental appliance on the model of the arch of the patient.
Clause 49. The method of clause 46, wherein modeling the retention of a first plurality of aligners on the model of the arch of the patient includes: simulating intrusive tooth movement on a first subset of teeth of the patient in the arch of the patient with a first aligner of the first plurality of aligners.
Clause 50. The method of clause 47, wherein modeling the retention of a first plurality of aligners on the model of the arch of the patient includes: simulating intrusive tooth movement on a second subset of teeth of the patient in the arch of the patient with a second aligner of the first plurality of aligners.
Clause 51. The method of clause 48, wherein modeling the retention of a first plurality of aligners on the model of the arch of the patient includes: determining an intrusion force for each of the first subset of teeth with the first of the first plurality of aligners and for each of the second subset of teeth with the second of the first plurality of aligners.
Clause 52. The method of clause 49, wherein modeling the retention of a first plurality of aligners on the model of the arch of the patient includes: normalizing the intrusion force for each of the first and second subset of teeth.
Clause 53. The method of clause 50, wherein modeling the retention of a second plurality of aligners on the retentive model of an arch of the patient includes: simulating intrusive tooth movement on a first subset of teeth of the patient in the arch of the patient with a first aligner of the second plurality of aligners.
Clause 54. The method of clause 51, wherein modeling the retention of a second plurality of aligners on the retentive model of an arch of the patient includes: simulating intrusive tooth movement on a second subset of teeth of the patient in the arch of the patient with a first aligner of the second plurality of aligners.
Clause 55. The method of clause 52, wherein modeling the retention of a first plurality of aligners on the model of the arch of the patient includes: determining an intrusion force for each of the first subset of teeth with the first of the first plurality of aligners and for each of the second subset of teeth with the second of the first plurality of aligners.
Clause 56. The method of clause 53, wherein modeling the retention of a first plurality of aligners on the model of the arch of the patient includes: normalizing the intrusion force for each of the first and second subset of teeth.
Clause 57. The method of clause 54, wherein generating a retention difference includes: subtracting on a tooth by tooth basis, a normalized intrusion force for the first and second subset of teeth with the first plurality of aligners from a normalized intrusion force for the first and second subset of teeth with the second plurality of aligners.
Clause 58. The method of clause 55, further comprising: selecting, based on the retention difference, one or more teeth with low retention, and wherein modifying the first aligner to increase retention includes modifying the first aligner at the one or more teeth or teeth adjacent the one or more teeth.
Clause 59. The method of clause 55, further comprising: selecting, based on the retention difference, one or more teeth with low retention, and wherein modifying the first aligner to increase retention includes modifying the first aligner at the one or more teeth and teeth adjacent the one or more teeth.
Clause 60. The method of any one of the preceding clauses, wherein modifying the first aligner includes: defining a trim line of the aligner along a gingival line that is offset from the gingival line by at least 1 mm with a radius of curvature of between 1 mm and 200 mm.
Clause 61. The method of any one of the preceding clauses, wherein modifying the first aligner includes: wherein the offset extends from a first tooth immediately adjacent the selected one or more teeth with low retention on a first side to a second tooth immediately adjacent the selected one or more teeth with low retention on a second side.
Clause 62. The method of any one of the preceding clauses, wherein modifying the first aligner includes: wherein the offset extends from a first and second tooth immediately adjacent the selected one or more teeth with low retention on a first side to a third and fourth tooth immediately adjacent the selected one or more teeth with low retention on a second side.
Clause 63. The method of any one of the preceding clauses, wherein modifying the first aligner includes: offsetting the size of one or more tooth receiving cavities by between −0.01 mm and −0.15 mm to reduce the size of the tooth receiving cavity.
Clause 64. The method of any one of the preceding clauses, wherein modifying the first aligner includes: offsetting the size of one or more tooth receiving cavities by between −0.02 mm and −0.8 mm to reduce the size of the tooth receiving cavity.
Clause 65. The method of any one of the preceding clauses, wherein a negative offset adjusts a location of sidewalls of the tooth receiving cavity towards the tooth.
Clause 66. The method of any one of the preceding clauses, wherein the one or more tooth receiving cavities include a tooth receiving cavities is immediately adjacent the selected one or more teeth.
Clause 67. The method of any one of the preceding clauses, wherein the one or more tooth receiving cavities include a first and second tooth receiving cavities immediately adjacent the selected one or more teeth with low retention on a first side and a third and fourth tooth immediately adjacent the selected one or more teeth with low retention on a second side.
Clause 68. A system for generating dental appliances, comprising: one or more processors and memory comprising instructions that when executed by one or more of the processors causes the system to carry out the method of any one of clauses the preceding clauses.
Clause 69. A method for designing dental appliances, comprising: receiving a 3D model of a patient's oral cavity; determining a target arrangement of teeth of the patient from the 3D model; generating a treatment plan for moving teeth of a patient from an initial position towards and final position over a plurality of stages; for a stage of the plurality of stages: identifying which of the teeth of the patient are stationary for the stage of the treatment plan; modifying movement of the stationary teeth for the stage of the treatment plan; revising the treatment plan using a modified movement of the stationary teeth for the stage of the plurality of stages to generate a revised treatment plan.
Clause 70. The method of clause 67, further comprising: generating appliance geometries based on the revised treatment plan.
Clause 71. The method of clause 68, further comprising: fabricating a set of aligners based on the appliance geometries.
Clause 72. The method of clause 67, wherein: revising the treatment plan includes providing the modified movement of the stationary teeth for the stage of the treatment plan a treatment management system.
Clause 73. The method of clause 68, wherein: generating appliance geometries includes providing the revised treatment plan to an appliance generation system.
Clause 74. The method of clause 71, further comprising: fabricating a set of aligners based on the appliance geometries of an oral cavity using one or more of an intraoral surface scan of the patient's oral cavity, impressions of the dentition of the patient, x-rays of the patient's oral cavity, CBCT scans of the patient's oral cavity, or 2D images of the dentition of the patient.
Clause 75. The method of clause 67, further comprising: segmenting the 3D model of the dentition of the patient to separate each tooth of the dentition of the patient from each other tooth of the dentition of the patient.
Clause 76. The method of clause 67, wherein determining one or more stationary teeth for a stage of the treatment plan includes: determining the one or more stationary teeth based on movement below a threshold in one or more degrees of freedom.
Clause 77. The method of clause 74, wherein the threshold is between 0 mm and 0.15 mm.
Clause 78. The method of clause 74, wherein the degree of freedom is along a buccal-lingual axis or a mesial-distal axis.
Clause 79. The method of clause 74, wherein modifying the movement of the one or more stationary teeth includes modifying the movement of the one or more stationary teeth in the degree of freedom.
Clause 80. The method of clause 74, wherein modifying the movement of the one or more stationary teeth includes modifying the movement of the one or more stationary teeth by distance between 0.01 mm and 0.15 mm in a first direction in the degree of freedom for the stage of the treatment plan.
Clause 81. The method of clause 78, further comprising: modifying the movement of the one or more stationary teeth in a next stage of treatment in a second direction, opposite the first direction in the degree of freedom.
Clause 82. The method of clause 78, further comprising: modifying the movement of the one or more stationary teeth in a next stage of treatment by the distance in a second direction, opposite the first direction in the degree of freedom.
Clause 83. The method of clause 67, further comprising: modifying movement of one or more stationary teeth in each stage of the treatment plan.
Clause 84. A system for designing dental appliances, the system comprising: one or more processors; memory comprising instructions that when executed cause the system to: receive a treatment plan for moving teeth of a patient from an initial position towards and final position and including a model of an arch of the patient; determine one or more stationary teeth for a stage of the treatment plan; modify movement of the one or more stationary teeth for the stage of the treatment plan to generate a revised treatment plan; and output the revised treatment plan.
Clause 85. The system of clause 82, further comprising: a fabrication machine, and wherein the memory further comprises instructions that when executed further cause the system to: fabricate a set of aligners based on the revised treatment plan.
Clause 86. The system of clause 82, wherein the instructions that when executed cause the system to determine one or more stationary teeth for a stage of the treatment plan further cause the system to: determine the one or more stationary teeth based on movement below a threshold in one or more degrees of freedom.
Clause 87. The system of clause 84, wherein the threshold is between 0 mm and 0.15 mm.
Clause 88. The system of clause 84, wherein the degree of freedom is along a buccal-lingual axis or a mesial-distal axis.
Clause 89. The system of clause 84, wherein the instructions that when executed cause the system to modify the movement of the one or more stationary teeth further cause the system to modify the movement of the one or more stationary teeth in the degree of freedom.
Clause 90. The system of clause 84, wherein instructions that when executed cause the system to modify the movement of the one or more stationary teeth further cause the system to modify the movement of the one or more stationary teeth by distance between 0.01 mm and 0.15 mm in a first direction in the degree of freedom for the stage of the treatment plan.
Clause 91. The system of clause 88, wherein the memory further comprises instructions that when executed further cause the system to:
Clause 92. modify the movement of the one or more stationary teeth in a next stage of treatment in a second direction, opposite the first direction in the degree of freedom.
Clause 93. The system of clause 88, wherein the memory further comprises instructions that when executed further cause the system to: modify the movement of the one or more stationary teeth in a next stage of treatment by the distance in a second direction, opposite the first direction in the degree of freedom.
Clause 94. The system of clause 82, wherein the memory further comprises instructions that when executed further cause the system to: modify movement of one or more stationary teeth in each stage of the treatment plan.
Clause 95. A system for generating dental appliances, comprising: one or more processors and memory comprising instructions that when executed by one or more of the processors causes the system to carry out the method of any one of clauses the preceding clauses.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof.

1. A method for designing dental appliances, comprising:
receiving a 3D model of a patient's oral cavity;
determining a target arrangement of teeth of the patient from the 3D model;
generating a treatment plan for moving teeth of a patient from an initial position towards and final position over a plurality of stages;
for a stage of the plurality of stages:
identifying which of the teeth of the patient are stationary for the stage of the treatment plan;
modifying movement of the stationary teeth for the stage of the treatment plan;
revising the treatment plan using a modified movement of the stationary teeth for the stage of the plurality of stages to generate a revised treatment plan.

2. The method of claim 1, further comprising:
generating appliance geometries based on the revised treatment plan; and
fabricating a set of aligners based on the appliance geometries.

3. The method of claim 1, wherein:
revising the treatment plan includes providing the modified movement of the stationary teeth for the stage of the treatment plan to a treatment management system.

4. The method of claim 2, wherein:
generating appliance geometries includes providing the revised treatment plan to an appliance generation system.

5. The method of claim 4, further comprising:
fabricating a set of aligners based on the appliance geometries of an oral cavity using one or more of an intraoral surface scan of the patient's oral cavity, impressions of the dentition of the patient, x-rays of the patient's oral cavity, CBCT scans of the patient's oral cavity, or 2D images of the dentition of the patient.

6. The method of claim 1, wherein determining one or more stationary teeth for a stage of the treatment plan includes:
determining the one or more stationary teeth based on movement below a threshold in one or more degrees of freedom.

7. The method of claim 6, wherein the threshold is between 0 mm and 0.15 mm.

8. The method of claim 6, wherein the degree of freedom is along a buccal-lingual axis or a mesial-distal axis.

9. The method of claim 6, wherein modifying the movement of the one or more stationary teeth includes modifying the movement of the one or more stationary teeth in the degree of freedom.

10. The method of claim 6, wherein modifying the movement of the one or more stationary teeth includes modifying the movement of the one or more stationary teeth by distance between 0.01 mm and 0.15 mm in a first direction in the degree of freedom for the stage of the treatment plan.

11. The method of claim 10, further comprising:
modifying the movement of the one or more stationary teeth in a next stage of treatment in a second direction, opposite the first direction in the degree of freedom.

12. The method of claim 10, further comprising:
modifying the movement of the one or more stationary teeth in a next stage of treatment by the distance in a second direction, opposite the first direction in the degree of freedom.

13. A system for designing dental appliances, the system comprising:
one or more processors;
memory comprising instructions that when executed cause the system to:
receive a treatment plan for moving teeth of a patient from an initial position towards and final position and including a model of an arch of the patient;
determine one or more stationary teeth for a stage of the treatment plan;
modify movement of the one or more stationary teeth for the stage of the treatment plan to generate a revised treatment plan;
output the revised treatment plan.

14. The system of claim 13, further comprising:
a fabrication machine, and wherein the memory further comprises instructions that when executed further cause the system to:
fabricate a set of aligners based on the revised treatment plan.

15. The system of claim 13, wherein the instructions that when executed cause the system to determine one or more stationary teeth for a stage of the treatment plan further cause the system to:
determine the one or more stationary teeth based on movement below a threshold in one or more degrees of freedom.

16. The system of claim 15, wherein the threshold is between 0 mm and 0.15 mm.

17. The system of claim 15, wherein the instructions that when executed cause the system to modify the movement of the one or more stationary teeth further cause the system to modify the movement of the one or more stationary teeth in the degree of freedom.

18. The system of claim 15, wherein instructions that when executed cause the system to modify the movement of the one or more stationary teeth further cause the system to modify the movement of the one or more stationary teeth by distance between 0.01 mm and 0.15 mm in a first direction in the degree of freedom for the stage of the treatment plan.

19. The system of claim 18, wherein the memory further comprises instructions that when executed further cause the system to:
modify the movement of the one or more stationary teeth in a next stage of treatment in a second direction, opposite the first direction in the degree of freedom.

20. The system of claim 18, wherein the memory further comprises instructions that when executed further cause the system to:
modify the movement of the one or more stationary teeth in a next stage of treatment by the distance in a second direction, opposite the first direction in the degree of freedom.
